(19) United States
(12) Patent Application Publication
(10) Pub. No.: US 2007/0088070 A1
(43) Pub. Date: Apr. 19, 2007

(54) Pyrazole derivatives, compositions containing such compounds and methods of use

(76) Inventors: Emma R. Parmee, Scotch Plains, NJ (US); Yusheng Xiong, Plainsboro, NJ (US); Jian Guo, Scotch Plains, NJ (US); Linda Brockunier, Orange, NJ (US)

Correspondence Address: MERCK AND CO., INC, P O BOX 2000, RAHWAY, NJ, 07065-0907, US

(21) Appl. No.: 11/583,253

(22) Filed: Oct. 19, 2006

Related U.S. Application Data
(60) Provisional application No. 60/728,177, filed on Oct. 19, 2005.

(51) Int. Cl. A61K 31/415; C07D 231/12

(52) U.S. Cl. 514/406; 548/377.1

(57) ABSTRACT

Pyrazoles having a naphthyl group attached are disclosed. The compounds are useful for treating type 2 diabetes and related conditions. Pharmaceutical compositions and methods of treatment are also included.

### BACKGROUND OF THE INVENTION

The present invention relates to pyrazole derivatives, compositions containing such compounds and various methods of treatment relating to type 2 diabetes mellitus and related conditions.

Diabetes refers to a disease process derived from multiple causative factors and is characterized by elevated levels of plasma glucose (hyperglycemia) in the fasting state or following glucose administration during an oral glucose tolerance test. Frank diabetes mellitus (e.g., a blood glucose level >126 mg/dL in a fasting state) is associated with increased and premature cardiovascular morbidity and mortality, and is related directly and indirectly to various metabolic conditions, including alterations of lipid, lipoprotein and apolipoprotein metabolism.

Patients with non-insulin dependent diabetes mellitus (type 2 diabetes mellitus), approximately 95% of patients with diabetes mellitus, frequently display elevated levels of serum lipids, such as cholesterol and triglycerides, and have poor blood-lipid profiles, with high levels of LDL-cholesterol and low levels of HDL-cholesterol. Those suffering from Type 2 diabetes mellitus are thus at an increased risk of developing macrovascular and microvascular complications, including coronary heart disease, stroke, peripheral vascular disease, hypertension (for example, blood pressure>130/80 mmHg in a resting state), nephropathy, neuropathy and retinopathy.

Patients having type 2 diabetes mellitus characteristically exhibit elevated plasma insulin levels compared with nondiabetic patients; these patients have developed a resistance to insulin stimulation of glucose and lipid metabolism in the main insulin-sensitive tissues (muscle, liver and adipose tissues). Thus, Type 2 diabetes, at least early in the natural progression of the disease is characterized primarily by insulin resistance rather than by a decrease in insulin production, resulting in insufficient uptake, oxidation and storage of glucose in muscle, inadequate repression of lipolysis in adipose tissue, and excess glucose production and secretion by the liver. The net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

Glucagon serves as the major regulatory hormone attenuating the effect of insulin in its inhibition of liver gluconeogenesis and is normally secreted by alpha cells in pancreatic islets in response to falling blood glucose levels. The hormone binds to specific receptors in liver cells that triggers glycogenolysis and an increase in gluconeogenesis through cAMP-mediated events. These responses generate glucose (e.g. hepatic glucose production) to help maintain euglycemia by preventing blood glucose levels from falling significantly. In addition to elevated levels of circulating insulin, type 2 diabetics have elevated levels of plasma glucagon and increased rates of hepatic glucose production. Antagonists of glucagon are useful in improving insulin responsiveness in the liver, decreasing the rate of gluconeogenesis and glycogenolysis, and lowering the rate of hepatic glucose output resulting in a decrease in the levels of plasma glucose.

### SUMMARY OF THE INVENTION

The present invention relates to a compound represented by formula I: 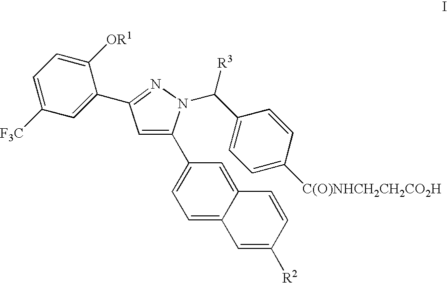
or a pharmaceutically acceptable salt or solvate thereof wherein:

R¹ represents C₁₋₆alkyl;

R² represents halo, C₁₋₃alkyl or OC₁₋₃alkyl and

R³ represents C₁₋₆alkyl.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched or cyclic, or combinations thereof, containing the indicated number of carbon atoms. Examples of alkyl groups include methyl, ethyl, propyl, isopropyl, butyl, sec- and tert-butyl, pentyl, hexyl. Cycloalkyl is a subset of alkyl. If no number of atoms is specified, 3-6 carbon atoms are intended, forming 1 carbocyclic ring. Examples of cycloalkyl include cyclopropyl, cyclobutyl, cyclopentyl and cyclohexyl.

“Halogen” (Halo) includes fluorine, chlorine, bromine and iodine, preferably chloro and fluoro, and more preferably chloro.

In its broadest aspect, the invention relates to a compound of formula I: 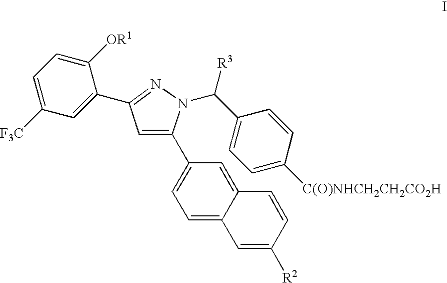
or a pharmaceutically acceptable salt or solvate thereof wherein:

R¹ represents C₁₋₆alkyl;

R² represents halo, C₁₋₃alkyl or OC₁₋₃alkyl and

R³ represents C₁₋₆alkyl.

One aspect of the invention that is of interest relates to compounds of formula I as described above wherein R¹ is selected from the group consisting of: methyl, ethyl, n-propyl, isopropyl, cyclopropyl, cyclopropylmethyl, cyclobutyl and cyclopentyl. Within this aspect of the invention, all other variables are as originally defined with respect to formula I.

Another aspect of the invention that is of interest relates to compounds of formula I as described above wherein R² is selected from the group consisting of: chloro, methyl, ethyl, propyl, isopropyl and methoxy. Within this aspect of the invention, all other variables are as originally defined with respect to formula I.

Another aspect of the invention that is of interest relates to compounds of formula I as described above wherein R³ is selected from the group consisting of: methyl, ethyl, n-propyl and n-butyl. Within this aspect of the invention, all other variables are as originally defined with respect to formula I.

Another aspect of the invention that is of even more interest relates to compounds of formula I as described above wherein: R¹ is selected from the group consisting of: methyl, ethyl, n-propyl, isopropyl, cyclopropyl, cyclopropylmethyl, cyclobutyl and cyclopentyl;

R² is selected from the group consisting of: chloro, methyl, ethyl, propyl, isopropyl and methoxy, and

R³ is selected from the group consisting of: methyl, ethyl, n-propyl and n-butyl. Within this aspect of the invention, all other variables are as originally defined with respect to formula I.

Examples of compounds that are of particular interest are set forth below:

TABLE 1

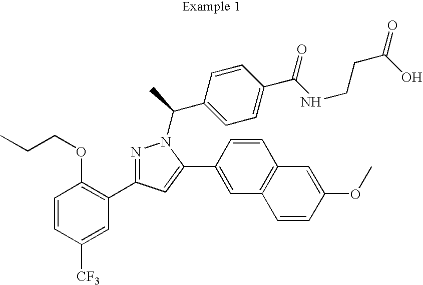

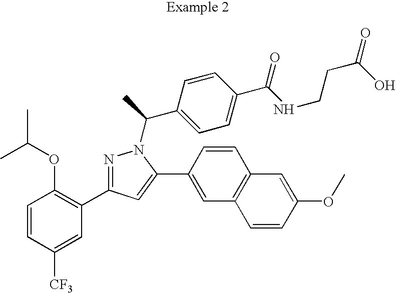

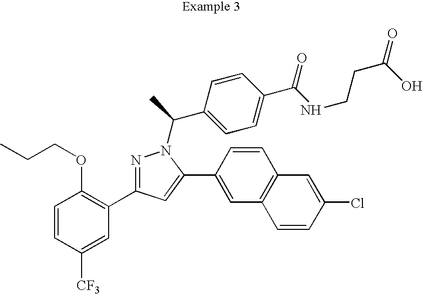

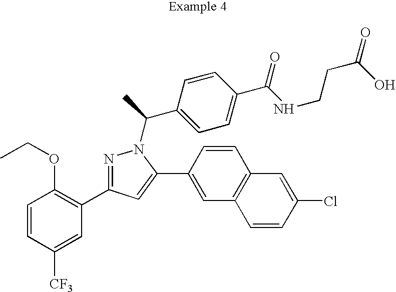

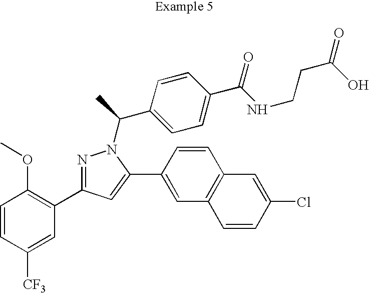

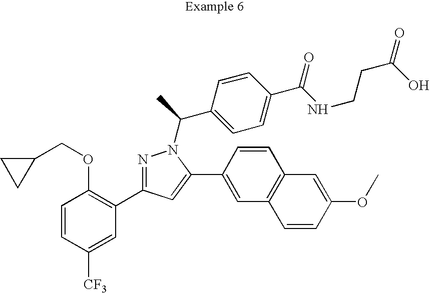

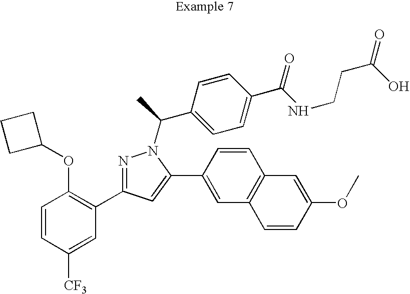

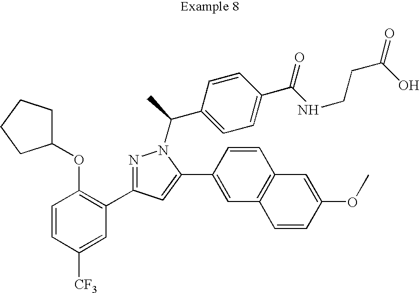

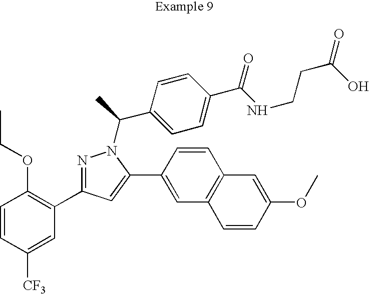

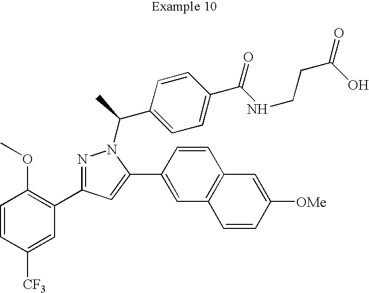

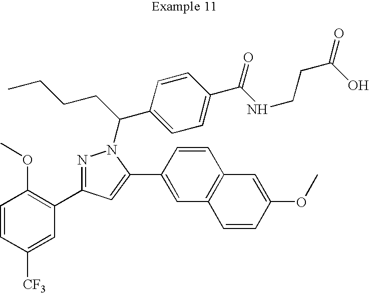

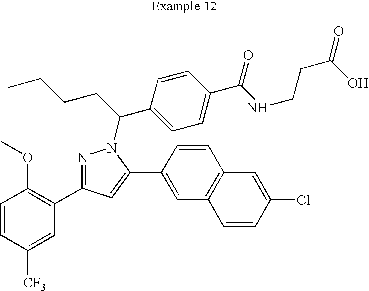

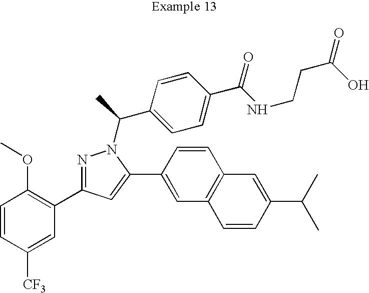

Another aspect of the invention that is of interest relates to a pharmaceutical composition comprising a compound as described above with respect to formula I in combination with a pharmaceutically acceptable carrier.

Another aspect of the invention that is of interest relates to a method of treating type 2 diabetes mellitus in a mammalian patient in need of such treatment comprising administering to said patient a compound as described above with respect to formula I in an amount that is effective to treat type 2 diabetes mellitus.

Another aspect of the invention that is of interest relates to a method of delaying the onset of type 2 diabetes mellitus in a mammalian patient in need thereof, comprising administering to the patient a compound as described above in accordance with formula I in an amount that is effective to delay the onset of type 2 diabetes mellitus.

Another aspect of the invention that is of interest relates to a method of treating hyperglycemia, diabetes or insulin resistance in a mammalian patient in need of such treatment which comprises administering to said patient a compound as described above in accordance with formula I in an amount that is effective to treat hyperglycemia, diabetes or insulin resistance.

Another aspect of the invention that is of interest relates to a method of treating non-insulin dependent diabetes mellitus in a mammalian patient in need of such treatment comprising administering to the patient an anti-diabetic effective amount of a compound in accordance with formula I as described above.

Another aspect of the invention that is of interest relates to a method of treating obesity in a mammalian patient in need of such treatment comprising administering to said patient a compound in accordance with formula I as described above in an amount that is effective to treat obesity.

Another aspect of the invention that is of interest relates to a method of treating Syndrome X in a mammalian patient in need of such treatment, comprising administering to said patient a compound in accordance with formula I as described above in an amount that is effective to treat Syndrome X.

Another aspect of the invention that is of interest relates to a method of treating a lipid disorder selected from the group consisting of dyslipidemia, hyperlipidemia, hypertriglyceridemia, hypercholesterolemia, low HDL and high LDL in a mammalian patient in need of such treatment, comprising administering to said patient a compound as described above with respect to formula I in an amount that is effective to treat said lipid disorder.

Another aspect of the invention that is of interest relates to a method of treating atherosclerosis in a mammalian patient in need of such treatment, comprising administering to said patient a compound in accordance with formula I as described above in an amount effective to treat atherosclerosis.

Another aspect of the invention that is of interest relates to a method of treating a condition selected from the group consisting of: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component, in a mammalian patient in need of such treatment, comprising administering to the patient a compound in accordance with formula I as described above in an amount that is effective to treat said condition.

Another aspect of the invention that is of interest relates to a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component in a mammalian patient in need of such treatment, comprising administering to the patient a compound in accordance with formula I as described above in an amount that is effective to delay the onset of said condition.

Another aspect of the invention that is of interest relates to a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component in a mammalian patient in need of such treatment, comprising administering to the patient a compound of formula I as described above in an amount that is effective to reduce the risk of developing said condition.

Another aspect of the invention that is of interest relates to a method of treating a condition selected from the group consisting of:

(1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (1.3) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component, in a mammalian patient in need of such treatment,

comprising administering to the patient effective amounts of a compound of formula I as described above, and a compound selected from the group consisting of:

(a) DPP-IV inhibitors, such as the compounds disclosed in U.S. Pat. No. 6,699,871B1 granted on Mar. 2, 2004, incorporated herein by reference; (b) insulin sensitizers selected from the group consisting of (i) PPAR agonists and (ii) biguanides; (c) insulin and insulin mimetics; (d) sulfonylureas and other insulin secretagogues; (e) alpha glucosidase inhibitors; (f) other glucagon receptor antagonists; (g) GLP-1, GLP-1 mimetics, and GLP-1 receptor agonists; (h) GIP,GIP mimetics, and GIP receptor agonists; (i) PACAP, PACAP mimetics, and PACAP receptor 3 agonists; (j) cholesterol lowering agents selected from the group consisting of (i) HMG-CoA reductase inhibitors, (ii) sequestrants, (iii) nicotinyl alcohol, nicotinic acid and salts thereof, (iv) PPAR alpha agonists, (v) PPAR alpha/gamma dual agonists, (vi) inhibitors of cholesterol absorption, (vii) acyl CoA:cholesterol acyltransferase inhibitors, (viii) anti-oxidants and (ix) LXR modulators; (k) PPAR delta agonists; (1) antiobesity compounds; (m) an ileal bile acid transporter inhibitor; (n) anti-inflammatory agents excluding glucocorticoids; (O) protein tyrosine phosphatase-1B (PTP-IB) inhibitors, and (p) CB1 antagonists/inverse agonists, such as rimonabant and those disclosed in WO03/077847A2, published on Sep. 25, 2003, and WO05/000809 published on Jan. 6, 2005, incorporated herein by reference,

said compounds being administered to the patient in amounts that are effective to treat said condition.

Another aspect of the invention that is of interest relates to a method of treating a condition selected from the group consisting of hypercholesterolemia, atherosclerosis, low HDL levels, high LDL levels, hyperlipidemia, hypertriglyceridemia and dyslipidemia, in a mammalian patient in need of such treatment, comprising administering to the patient therapeutically effective amounts of a compound of formula I as described above and an HMG-CoA reductase inhibitor.

More particularly, another aspect of the invention that is of interest relates to a method of treating a condition selected from the group consisting of hypercholesterolemia, atherosclerosis, low HDL levels, high LDL levels, hyperlipidemia, hypertriglyceridemia and dyslipidemia, in a mammalian patient in need of such treatment, comprising administering to the patient therapeutically effective amounts of a compound of formula I as described above and an HMG-CoA reductase inhibitor wherein the HMG-CoA reductase inhibitor is a statin.

Even more particularly, another aspect of the invention that is of interest relates to a method of treating a condition selected from the group consisting of hypercholesterolemia, atherosclerosis, low HDL levels, high LDL levels, hyperlipidemia, hypertriglyceridemia and dyslipidemia, in a mammalian patient in need of such treatment, comprising administering to the patient therapeutically effective amounts of a compound of formula I as described above and an HMG-CoA reductase inhibitor, wherein the HMG CoA reductase inhibitor is a statin selected from the group consisting of lovastatin, simvastatin, pravastatin, fluvastatin, atorvastatin, itavastatin, ZD-4522 and rivastatin.

Another aspect of the invention that is of interest relates to a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions comprising administering to a mammalian patient in need of such treatment therapeutically effective amounts of a compound of formula I as described above and an HMG-CoA reductase inhibitor.

Another aspect of the invention that is of interest relates to a method for delaying the onset or reducing the risk of developing atherosclerosis in a human patient in need of such treatment comprising administering to said patient effective amounts of a compound of formula I as described above and an HMG-CoA reductase inhibitor.

More particularly, another aspect of the invention that is of interest relates to a method for delaying the onset of, or reducing the risk of developing atherosclerosis in a human patient in need of such treatment comprising administering to said patient effective amounts of a compound of formula I as described above and an HMG-CoA reductase inhibitor wherein the HMG-CoA reductase inhibitor is a statin.

Even more particularly, another aspect of the invention that is of interest relates to a method for delaying the onset or reducing the risk of developing atherosclerosis in a human patient in need of such treatment comprising administering to said patient effective amounts of a compound of formula I as described above and an HMG-CoA reductase inhibitor wherein the HMG-CoA reductase inhibitor is a statin selected from the group consisting of: lovastatin, simvastatin, pravastatin, fluvastatin, atorvastatin, itavastatin, ZD-4522 and rivastatin.

Yet even more particularly, another aspect of the invention that is of interest relates to a method for delaying the onset or reducing the risk of developing atherosclerosis in a human patient in need of such treatment comprising administering to said patient effective amounts of a compound of formula I as described above and an HMG-CoA reductase inhibitor wherein the HMG-CoA reductase inhibitor is simvastatin.

Another aspect of the invention that is of interest relates to a method for delaying the onset or reducing the risk of developing atherosclerosis in a human patient in need of such treatment comprising administering to said patient effective amounts of a compound of formula I as described above and a cholesterol absorption inhibitor. More particularly, another aspect of the invention that is of interest relates to a method for delaying the onset or reducing the risk of developing atherosclerosis in a human patient in need of such treatment comprising administering to said patient effective amounts of a compound of formula I as described above and a cholesterol absorption inhibitor wherein the cholesterol absorption inhibitor is ezetimibe.

Another aspect of the invention that is of interest relates to a method for delaying the onset or reducing the risk of developing the other diseases and conditions mentioned above, in a mammalian patient in need of such treatment comprising administering to said patient effective amounts of a compound of formula I as described above, and a cholesterol absorption inhibitor.

More particularly, another aspect of the invention that is of interest relates to a method for delaying the onset or reducing the risk of developing the other diseases and conditions mentioned above, in a human patient in need of such treatment comprising administering to said patient effective amounts of a compound of formula I as described above, and a cholesterol absorption inhibitor, wherein the cholesterol absorption inhibitor is ezetimibe.

Another aspect of the invention that is of interest relates to a pharmaceutical composition comprising (1) a compound of formula I as described above; (2) a compound selected from the group consisting of: (a) DPP-IV inhibitors, such as those disclosed in U.S. Pat. No. 6,699,871B1 granted on Mar. 2, 2004; (b) insulin sensitizers selected from the group consisting of (i) PPAR agonists and (ii) biguanides; (c) insulin and insulin mimetics; (d) sulfonylureas and other insulin secretagogues; (e) alpha glucosidase inhibitors; (f) other glucagon receptor antagonists; (g) GLP-1, GLP-1 mimetics and GLP-1 receptor agonists; (h) GIP, GIP mimetics and GIP receptor agonists; (i) PACAP, PACAP mimetics, and PACAP receptor 3 agonists; (j) cholesterol lowering agents selected from the group consisting of (i) HMG-CoA reductase inhibitors, (ii) sequestrants, (iii) nicotinyl alcohol, nicotinic acid or a salt thereof, (iv) PPAR alpha agonists, (v) PPAR alpha/gamma dual agonists, (vi) inhibitors of cholesterol absorption, (vii) acyl CoA:cholesterol acyltransferase inhibitors, (viii) anti-oxidants and (ix) LXR modulators; (k) PPAR delta agonists; (1) antiobesity compounds; (m) an ileal bile acid transporter inhibitor; (n) anti-inflammatory agents other than glucocorticoids; (O) protein tyrosine phosphatase-1B (PTP-1B) inhibitors; and (p) CB1 antagonist/inverse agonists, such as rimonabant, and those disclosed in WO03/077847A2 published on Sep. 25, 2003 and WO05/000809 published on Jan. 6, 2005, and (3) a pharmaceutically acceptable carrier.

One pharmaceutical composition that is of interest is comprised of a compound of formula I as described herein, or a pharmaceutically acceptable salt or solvate thereof, in combination with a DPP-IV inhibitor selected from the group consisting of: 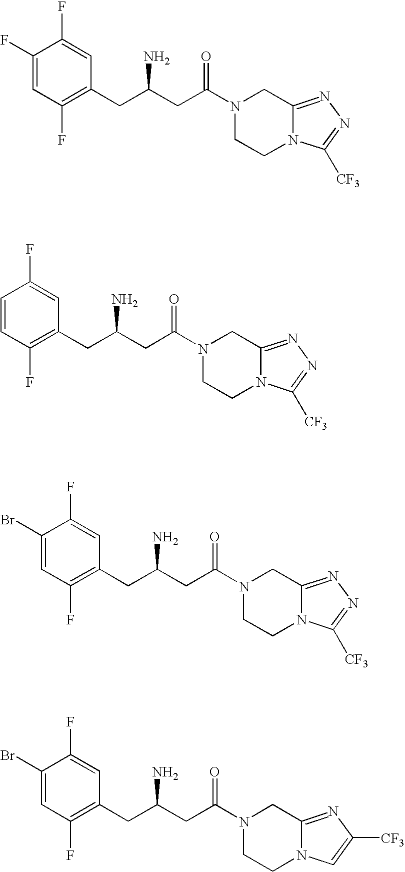
or a pharmaceutically acceptable salt or solvate thereof in combination with a pharmaceutically acceptable carrier.

Another pharmaceutical composition that is of particular interest is comprised of a compound of formula I as described herein, or a pharmaceutically acceptable salt or solvate thereof, in combination with a CB1 receptor antagonist/inverse agonist, in combination with a pharmaceutically acceptable carrier. Examples of CB1 antagonist/inverse agonists that are of particular interest in the invention described herein include rimonabant, the following which are disclosed in WO03/077847A2 published on Sep. 25, 2003:
(1) N-[3-(4-chlorophenyl)-1-methyl-2-phenylpropyl]-2-(4-chlorophenyloxy)-2-methylpropanamide;
(2) N-[3-(4-chlorophenyl)-1-methyl-2-phenylpropyl]-2-(2-pyridyloxy)-2-methylpropanamide;
(3) N-[3-(4-chlorophenyl)-1-methyl-2-(3-pyridyl)propyl]-2-(4-chlorophenyloxy)-2-methylpropanamide;
(4) N-[3-(4-chlorophenyl)-1-methyl-2-phenylpropyl]-2-(3,5-difluorophenyloxy)-2-methylpropanamide;
(5) N-[3-(4-chlorophenyl)-2-phenyl-1-methylpropyl]-2-(3,5-dichlorophenyloxy)-2-methylpropanamide;
(6) N-[3-(4-chlorophenyl)-1-methyl-2-phenylpropyl]-2-(3-chlorophenyloxy)-2-methylpropanamide;
(7) N-[3-(4-chlorophenyl)-2-(3,5-difluorophenyl)-1-methylpropyl]-2-(2-pyridyloxy)-2-methylpropanamide;
(8) N-[3-(4-chlorophenyl)-1-methyl-2-phenyl-propyl]-2-(5-chloro-2-pyridyloxy)-2-methylpropanamide;
(9) N-[3-(4-chlorophenyl)-1-methyl-2-phenylpropyl]-2-(6-methyl-pyridyloxy)-2-methylpropanamide;
(10) N-[3-(4-chlorophenyl)-1-methyl-2-phenylpropyl]-2-(phenyloxy)-2-methylpropanamide;
(11) N-[(3-(4-chlorophenyl)-1-methyl-2-phenylpropyl]-2-(5-trifluoromethylpyridyloxy)-2-methylpropanamide;
(12) N-[3-(4-chlorophenyl)-2-(3-pyridyl)-1-methylpropyl]-2-(5-trifluoromethyl-2-pyridyloxy)-2-methylpropanamide;
(13) N-[3-(4-chlorophenyl)-2-(3-cyanophenyl)-1-methylpropyl]-2-(5-trifluoromethyl-2-pyridyloxy)-2-methylpropanamide;
(14) N-[3-(4-chlorophenyl)-2-(5-chloro-3-pyridyl)-1-methylpropyl]-2-(5-trifluoromethyl-2-pyridyloxy)-2-methylpropanamide;
(15) N-[3-(4-chlorophenyl)-2-(5-methyl-3-pyridyl)-1-methylpropyl]-2-(5-trifluoromethyl-2-pyridyloxy)-2-methylpropanamide;
(16) N-[3-(4-chlorophenyl)-2-(5-cyano-3-pyridyl)-1-methylpropyl]-2-(5-trifluoromethyl-2-pyridyloxy)-2-methylpropanamide;
(17) N-[3-(4-chlorophenyl)-2-(3-methylphenyl)-1-methylpropyl]-2-(5-trifluoromethyl-2-pyridyloxy)-2-methylpropanamide;
(18) N-[3-(4-chlorophenyl)-2-phenyl-1-methylpropyl]-2-(4-trifluoromethyl-2-pyridyloxy)-2-methylpropanamide;
(19) N-[3-(4-chlorophenyl)-2-phenyl-1-methylpropyl]-2-(4-trifluoromethyl-2-pyrimidyloxy)-2-methylpropanamide;
(20) N-[3-(4-chlorophenyl)-1-methyl-2-(thiophen-3-yl)propyl]-2-(5-chloro-2-pyridyloxy)-2-methylpropanamide;
(21) N-[3-(5-chloro-2-pyridyl)-2-phenyl-1-methylpropyl]-2-(5-trifluoromethyl-2-pyridyloxy)-2-methylpropanamide;
(22) N-[3-(4-methyl-phenyl)-1-methyl-2-phenylpropyl]-2-(4-trifluoromethyl-phenyloxy)-2-methylpropanamide;
(23) N-[3-(4-fluoro-phenyl)-2-(3-cyano-phenyl)-1-methylpropyl]-2-(5-trifluoromethyl-2-pyridyloxy)-2-methylpropanamide;
(24) N-[3-(4-chlorophenyl)-2-(1-indolyl)-1-methyl)propyl]-2-(5-trifluoromethyl-2-oxypyridine-2-yl)-2-methylpropanamide;
(25) N-[3-(4-chlorophenyl)-2-(7-azaindol-N-yl)-1-methyl)propyl]-2-(5-trifluoromethyl-2-pyridyloxy)-2-methylpropanamide;
(26) N-[3-(4-chloro-phenyl)-2-(1-indolinyl)-1-methylpropyl]-2-(5-trifluoromethyl-2-pyridyloxy)-2-methylpropanamide;
(27) N-[3-(4-chloro-phenyl)-2-(N-methyl-anilino)-1-methylpropyl]-2-(5-trifluoromethyl-2-pyridyloxy)-2-methylpropanamide;
(28) N-[3-(4-methoxy-phenyl)-2-(3-cyano-phenyl)-1-methylpropyl]-2-(5-trifluoromethyl-2-pyridyloxy)-2-methylpropanamide;
(29) N-[3-(4-chlorophenyl)-2-(3-cyanophenyl)-1-methylpropyl]-2-(6-trifluoromethyl-4-pyrimidyloxy)-2-methylpropanamide;
(30) N-[2-(3-cyanophenyl)-1,4-dimethylpentyl]-2-(5-trifluoromethyl-2-pyridyloxy)-2-methylpropanamide;
(31) N-[3-(4-chlorophenyl)-2-(1-oxido-5-cyano-3-pyridyl]-1-methylpropyl]-2-(5-trifluoromethyl-2-pyridyloxy)-2-methylpropanamide;
(32) N-[2-(3-cyanophenyl)-3-cyclobutyl-1-methylpropyl]-2-(5-trifluoromethyl-2-pyridyloxy)-2-methylpropanamide;
(33) N-[2-(3-cyanophenyl)-1-methyl-heptyl]-2-(5-trifluoromethyl-2-pyridyloxy)-2-methylpropanamide;
(34) N-[2-(3-cyanophenyl)-3-cyclopentyl-1-methylpropyl]-2-(5-trifluoromethyl-2-pyridyloxy)-2-methylpropanamide;
(35) N-[2-(3-cyanophenyl)-3-cyclohexyl-1-methylpropyl]-2-(5-trifluoromethyl-2-pyridyloxy)-2-methylpropanamide;
   and in WO05/000809 published on Jan. 6, 2005, which includes the following:
3-{1-[Bis(4-chlorophenyl)methyl]azetidin-3-ylidene}-3-(3,5-difluorophenyl)-2,2-dimethylpropanenitrile 1-{1-[1-(4-chlorophenyl)pentyl]azetidin-3-yl}-1-(3,5-difluorophenyl)-2-methylpropan-2-ol 3-((S)-(4-chlorophenyl) {3-[(1S)-1-(3,5-difluorophenyl)-2-hydroxy-2-methylpropyl]azetidin-1-yl}methyl)benzonitrile
3-((S)-(4-chlorophenyl) {3-[(1S)-1-(3,5-difluorophenyl)-2-fluoro-2-methylpropyl]azetidin-1-yl}methyl)benzonitrile
3-((4-chlorophenyl) {3-[1-(3,5-difluorophenyl)-2,2-dimethylpropyl]azetidin-1-yl}methyl)benzonitrile
3-((1S)-1-{1-[(S)-(3-cyanophenyl)(4-cyanophenyl)methyl]azetidin-3-yl}-2-fluoro-2-methylpropyl)-5-fluorobenzonitrile
3-[(S)-(4-chlorophenyl)(3-{(1S)-2-fluoro-1-[3-fluoro-5-(4H-1,2,4-triazol-4-yl)phenyl]-2-methylpropyl}azetidin-1-yl)methyl]benzonitrile and
5-((4-chlorophenyl) {3-[(1S)-1-(3,5-difluorophenyl)-2-fluoro-2-methylpropyl]azetidin-1-yl}methyl)thiophene-3-carbonitrile,
   as well as the pharmaceutically acceptable salts and solvates thereof, in combination with a pharmaceutically acceptable carrier.
   Optical Isomers—Diastereomers—Geometric Isomers—Tautomers

Many of the compounds of formula I contain one or more asymmetric centers and thus occur as racemates and racemic mixtures, single enantiomers, diastereomeric mixtures and individual diastereomers. The present invention includes all such isomeric forms of the compounds, in pure form as well as in mixtures.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixtures thereof are encompassed with the compounds of Formula I.

Salts and Solvates

Salts and solvates of compounds of formula I are included in the present invention. The term “pharmaceutically acceptable salts” refers to salts prepared from pharmaceutically acceptable substantially non-toxic bases or acids including inorganic or organic bases and inorganic or organic acids, as well as salts that can be converted into pharmaceutically acceptable salts. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Particularly preferred are citric, hydrobromic, hydrochloric, maleic, phosphoric, sulfuric, and tartaric acids.

Solvates as used herein refers to the compound of formula I or a salt thereof, in association with a solvent, such as water. Representative examples include hydrates, hemihydrates, trihydrates and the like.

References to the compounds of Formula I are intended to include the pharmaceutically acceptable salts and solvates.

This invention relates to a method of antagonizing or inhibiting the production or activity of glucagon, thereby reducing the rate of gluconeogenesis and glycogenolysis, and the concentration of glucose in plasma.

The compounds of formula I can be used in the manufacture of a medicament for the prophylactic or therapeutic treatment of disease states in mammals associated with elevated levels of glucose, comprised of combining the compound of formula I with the carrier materials to provide the medicament.

Dose Ranges

The prophylactic or therapeutic dose of a compound of formula I will, of course, vary with the nature or severity of the condition to be treated, the particular compound selected and its route of administration. It will also vary according to the age, weight and response of the individual patient. In general, the daily dose range lies within the range of from about 0.001 mg to about 100 mg per kg body weight, preferably about 0.01 mg to about 50 mg per kg, and more preferably 0.1 to 10 mg per kg, in single or divided doses. It may be necessary to use dosages outside of these limits in some cases. The terms “effective amount”, “anti-diabetic effective amount” and the other terms appearing throughout the application addressing the amount of the compound to be used refer to the dosage ranges provided, taking into account any necessary variation outside of these ranges, as determined by the skilled physician.

Representative dosages of compounds of formula I, as well as the pharmaceutically acceptable salts and solvates thereof, for adults range from about 0.1 mg to about 1.0 g per day, preferably about 1 mg to about 500 mg, in single or divided doses. Representative dosages of compounds used in combination with the compounds of formula I are known, or the determination thereof is within the level of skill in the art, taking into account the description provided herein.

When intravenous or oral administration is employed, a representative dosage range is from about 0.001 mg to about 100 mg (preferably from 0.01 mg to about 10 mg) of a compound of Formula I per kg of body weight per day, and more preferably, about 0.1 mg to about 10 mg of a compound of formula I per kg of body weight per day.

When used in combination with other agents, the dosages noted above for the glucagon antagonist are provided along with the usual dose for the other medication. For example, when a DPP-IV inhibitor such as those disclosed in U.S. Pat. No. 6,699,871B1, is included, the DPP-IV inhibitor can be used in an amount ranging from about 11.0 mg to as high as about 1000 mg, preferably about 2.5 mg to about 250 mg, and in particular, about 50 mg or about 100 mg administered in single daily doses or in divided doses as appropriate. Similarly, when the glucagon antagonist is used in combination with a CB1 antagonist/inverse agonist, the CB 1 antagonist/inverse agonist can be used in an amount ranging from as low as about 0.1 mg to as high as about 1000 mg, more particularly, in an amount ranging from about 1.0 mg to about 100 mg, and even more particularly, in an amount from about 1.0 mg to about 10 mg, administered in single daily doses or in divided doses as appropriate. Examples of doses of CB 1 antagonist/inverse agonist include 1 mg, 2 mg, 3 mg, 4 mg, 5 mg, 6 mg, 7 mg, 8 mg, 9 mg and 10 mg.

Pharmaceutical Compositions

As mentioned above, the pharmaceutical composition comprises a compound of Formula I or a pharmaceutically acceptable salt or solvate thereof and a pharmaceutically acceptable carrier. The term “composition” encompasses a product comprising the active and inert ingredient(s), (pharmaceutically acceptable excipients) that make up the carrier, as well as any product which results, directly or indirectly, from the combination, complexation or aggregation of any two or more of the ingredients, or from dissociation of one or more of the ingredients, or from other types of reactions or interactions between ingredients. Preferably the composition is comprised of a compound of formula I in an amount that is effective to treat, prevent or delay the onset of type 2 diabetes mellitus, in combination with the pharmaceutically acceptable carrier.

Any suitable route of administration may be employed for providing a mammal, especially a human with an effective dosage of a compound of the present invention. For example, oral, rectal, topical, parenteral, ocular, pulmonary, nasal, and the like may be employed. Examples of dosage forms include tablets, troches, dispersions, suspensions, solutions, capsules, creams, ointments, aerosols and the like, with oral tablets being preferred.

In preparing oral compositions, any of the usual pharmaceutical media may be employed, such as, for example, water, glycols, oils, alcohols, flavoring agents, preservatives, coloring agents and the like, in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets. Solid oral preparations are preferred. Because of their ease of administration, tablets and capsules represent the most advantageous oral dosage unit forms. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any acceptable pharmaceutical process. All such methods include the step of combining the active ingredient(s) with the carrier components. In general, the compositions are prepared by uniformly and intimately admixing the active ingredient(s) with a liquid or finely divided solid carrier component, and then, if necessary, manipulating the blend into the desired product form. For example, a tablet may be prepared by compression or molding. Compressed tablets may be prepared by compressing free-flowing powder or granules, containing the active(s) optionally mixed with one or more excipients, e.g., binders, lubricants, diluents, surfactants and dispersants. Molded tablets may be made by molding a mixture of the powdered compound moistened with an inert liquid. Desirably, each tablet may contain, for example, from about 0.1 mg to about 1.0 g of the active ingredient and each cachet or capsule contains from about 0.1 mg to about 500 mg of the active ingredient.

The following are examples of pharmaceutical dosage forms containing a compound of Formula I:

| Injectable | | | | | |
|---|---|---|---|---|---|
| Suspension (im.) | mg/mL | | Tablet | Mg/tablet | |
| Compound of | 10.0 | | Compound of Formula 1 | 25.0 | |
| Formula 1 | | | | | |
| Methylcellulose | 5.0 | | Microcrystalline Cellulose | 415 | |
| Tween 80 | 0.5 | | Povidone | 14.0 | |
| Benzyl alcohol | 9.0 | | Pregelatinized Starch | 4.35 | |
| Benzalkonium | 1.0 | | Magnesium Stearate | 2.5 | |
| chloride | | | | | |
| Water for | t.d. 1.0 | mL | Total | 500 | mg |
| injection | | | | | |

| | mg/ | | | | |
|---|---|---|---|---|---|
| Capsule | capsule | | Aerosol | Per Canister | |
| Compound of | 25.0 | | Compound of | 250 | mg |
| Formula 1 | | | Formula 1 | | |
| Lactose | 735 | | Lecithin, NF | 1.2 | mg |
| | | | Liq. Conc. | | |
| Mg Stearate | 1.5 | | Trichloromethane, | 4.025 | g |
| | | | NF | | |
| Total | 600 | mg | Dichlorodifluoromethane, NF | 12.15 | g |

Combination Therapy

As previously described, the compounds of Formula I may be used in combination with other drugs that are used in the treatment/prevention/delaying the onset of type 2 diabetes mellitus, as well as other diseases and conditions described herein, for which compounds of Formula I are useful. Other drugs may be administered, by a route and in an amount commonly used, contemporaneously or sequentially with a compound of Formula I. When a compound of Formula I is used contemporaneously with one or more other drugs, a combination pharmaceutical composition containing such other drugs in addition to the compound of Formula I is preferred. Accordingly, the pharmaceutical compositions of the present invention include those that alternatively contain one or more other active ingredients, in addition to a compound of Formula I. Examples of other active ingredients that may be combined with a compound of Formula I, either administered separately or in the same pharmaceutical compositions, include, but are not limited to: (a) biguanides (e.g., buformin, metformin, phenformin), (b) PPAR agonists (e.g., troglitazone, pioglitazone, rosiglitazone), (c) insulin, (d) somatostatin, (e) alpha-glucosidase inhibitors (e.g., voglibose, miglitol, acarbose), (f) DPP-IV inhibitors, such as those disclosed in U.S. Pat. No. 6,699,871B1 granted on Mar. 2, 2004 (g) LXR modulators and (h) insulin secretagogues (e.g., acetohexamide, carbutamide, chlorpropamide, glibomuride, gliclazide, glimerpiride, glipizide, gliquidine, glisoxepid, glyburide, glyhexamide, glypinamide, phenbutamide, tolazamide, tolbutamide, tolcyclamide, nateglinide and repaglinide), and CB1 inhibitors, such as rimonabant and those compounds disclosed in WO03/077847A2 published on Sep. 25, 2003 and in WO05/000809 A1 published on Jan. 6, 2005.

The weight ratio of the compound of the Formula I to the second active ingredient may be varied within wide limits and depends upon the effective dose of each active ingredient. Generally, an effective dose of each will be used. Thus, for example, when a compound of the Formula I is combined with a PPAR agonist the weight ratio of the compound of the Formula I to the PPAR agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

For combination products, the compound of formula I may be combined with any other active ingredients and then added to the carrier ingredients; alternatively the order of mixing may be varied.

Examples of pharmaceutical combination compositions include: (1) a compound according to formula I, (2) a compound selected from the group consisting of: (a) DPP-IV inhibitors; (b) insulin sensitizers selected from the group consisting of (i) PPAR agonists and (ii) biguanides; (c) insulin and insulin mimetics; (d) sulfonylureas and other insulin secretagogues; (e) a-glucosidase inhibitors; (f) CB1 receptor antagonists/inverse agonists; (g) GLP-1, GLP-1 mimetics, and GLP-1 receptor agonists; (h) GIP, GIP mimetics, and GIP receptor agonists; (i) PACAP, PACAP mimetics, and PACAP receptor 3 agonists; (O) cholesterol lowering agents selected from the group consisting of (i) HMG-CoA reductase inhibitors, (ii) sequestrants, (iii) nicotinyl alcohol, nicotinic acid or a salt thereof, (iv) PPAR alpha agonists, (v) PPAR alpha/gamma dual agonists, (vi) inhibitors of cholesterol absorption, (vii) acyl CoA:cholesterol acyltransferase inhibitors, (viii) anti-oxidants and (ix) LXR modulators; (k) PPAR delta agonists; (1) antiobesity compounds; (m) an ileal bile acid transporter inhibitor; (n) anti-inflammatory agents other than glucocorticoids; and (O) protein tyrosine phosphatase-1B (PTP-1B) inhibitors; (p) CB1 antagonist/inverse agonists and (3) a pharmaceutically acceptable carrier.

The compounds of formula I can be synthesized in accordance with the general schemes provided below, taking into account the specific examples that are provided. Throughout the synthesis schemes, abbreviations are used with the following meanings unless otherwise indicated:

| | |
|---|---|
| Bu = butyl, t-Bu = t-butyl | Bn and Bnzl = benzyl |
| BOC, Boc = t-butyloxycarbonyl | CBZ, Cbz = Benzyloxycarbonyl |
| COD = cyclooctadiene | DCM = dichloromethane |
| CDI = carbonyl diimidazole | DIAD = diisopropylazodicarboxylate |
| DCC = Dicyclohexylcarbodiimide | DMAP = 4-Dimethylaminopyridine |
| DIEA = diisopropylethylamine | DMPU = 1,3-dimethy-3,4,5,6-tetrahydro- |
| | 2(1H)pyrimidinone |
| DMAC = dimethylacetamide | EtOH = ethanol |
| DMF = N,N-dimethylformamide | FAB-mass spectrum = Fast atom bombardment-mass |
| | spectroscopy |
| EtOAc = ethyl acetate | HPLC = High pressure liquid chromatography |
| eq. = equivalent(s) | LAH = Lithium aluminum hydride |
| HOAc = acetic acid | MTBE = methyl t-butyl ether |
| HOBT = N-hydroxybenztriazole | MeCN, CH₃CN = acetonitrile |
| MeOH = methanol | TFA = Trifluoroacetic acid |
| Me = methyl | NMe₂ = dimethylamino |
| PBS = phosphate buffer saline | 2ClPh = 2-chlorophenyl |
| Ph = phenyl | IPA = isopropanol |
| THF = Tetrahydrofuran | Py, Pyr = pyridyl |
| C₆H₁₁ = cyclohexyl | iPAc = isopropyl acetate |
| iPr = isopropyl | RT = room temperature |
| 2,4-diClPh = 2,4-dichlorophenyl | TMAD = tetramethylazodicarboxamide |

Compounds of the present invention may be prepared according to the methodology outlined in the following general synthetic schemes.

In one embodiment of the present invention, shown in Scheme 1, the compounds (I) may be prepared from intermediates 1 (vide infra) where R¹, R² and R³ are defined as above. The alkylation of 1 can be accomplished by treatment with an alkyl halide in the presence of a mild base such as K₂CO₃, or Cs₂CO₃, in a polar solvent like acetone, dimethylformamide, or dimethylsulfoxide, at temperatures between 20 to 100° C. This alkylation can also be realized through a Mitsunobu reaction (see Mitsunobu, O. Synthesis, 1981, 1) of 1 with alkyl alcohols in the presence of tributylphosphine and tetramethyl azodicarboxamide in toluene. Final products may then be obtained after conversion of the carboxylic ester to carboxylic acid. Removal of the ester when R=Me or Et is accomplished by saponification using a base such as aqueous lithium or sodium hydroxide in a polar solvent such as tetrahydrofuran, methanol, ethanol or a mixture of similar solvents. When R is a tert-butyl ester it is most conveniently removed by treatment with trifluoroacetic acid in methylene chloride for 0.5-3 h at ambient temperature. The product is purified from unwanted side products by recrystallization, trituration, preparative thin layer chromatography, flash chromatography on silica gel as described by W. C. Still et al, J. Org. Chem., 43, 2923, (1978), or reverse phase HPLC.

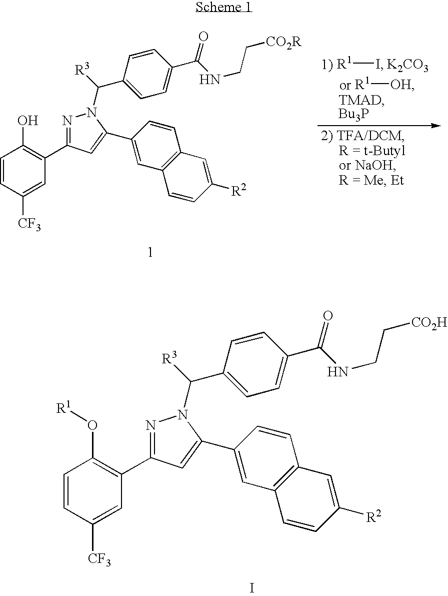

Compounds 1, can be prepared in a multi-step sequence starting from the cyclization of the β-ketoester 8 with the hydrazine 7, Scheme 2. Condensation of the β-ketoester 8 and benzyl hydrazine 7 can be carried out by heating the two components in a solvent such as acetic acid or acetonitrile for 1-8 h to give the pyrazolone 6. Pyrazolone 6 is then treated with triflic anhydride in a solvent such as DCM in the presence of a base such as triethylamine or pyridine at −78° C. to room temperature to afford the pyrazole-5-triflate intermediate, which upon removal of the methyl group by treatment with boron tribromide in DCM provides the intermediate 5. The triflate 5 can be coupled with boronic acid 4 using a palladium catalyst such as palladium tetrakis(triphenylphosphine), or palladium bis[-(di-t-butylphosphino)biphenyl] or palladium dichloride(dppf). The solvent is generally either dimethoxyethane, ethanol or toluene, and a base such as triethylamine, cesium or sodium carbonate or potassium fluoride is also added to the reaction, which may also contain water and is performed at elevated temperatures and may be carried out in a microwave reactor (see Wang et al., Tet. Lett., 2000, 41, 4713 for related cross-coupling reactions).

Elaboration at this point to β-alanine ester 1 can be achieved by saponification of the ester 3 using a base such as aqueous lithium or sodium hydroxide in a polar solvent such as tetrahydrofuran, dioxane, methanol, ethanol or a mixture of similar solvents. Coupling with the beta alanine ester is then achieved using 1-ethyl-3-(3-dimethylaminopropyl)-carbodiimide (EDC) and 1-hydroxybenzotriazole (HOBt) or benzotriazole-1-yloxytrispyrrolidinophosphonium hexafluorophosphate (PyBOP) and a base, generally diisopropylethylamine, in a solvent such as N,N-dimethylformamide (DMF) or methylene chloride for 0.5 to 48 hours at ambient temperature to yield the compound 1.

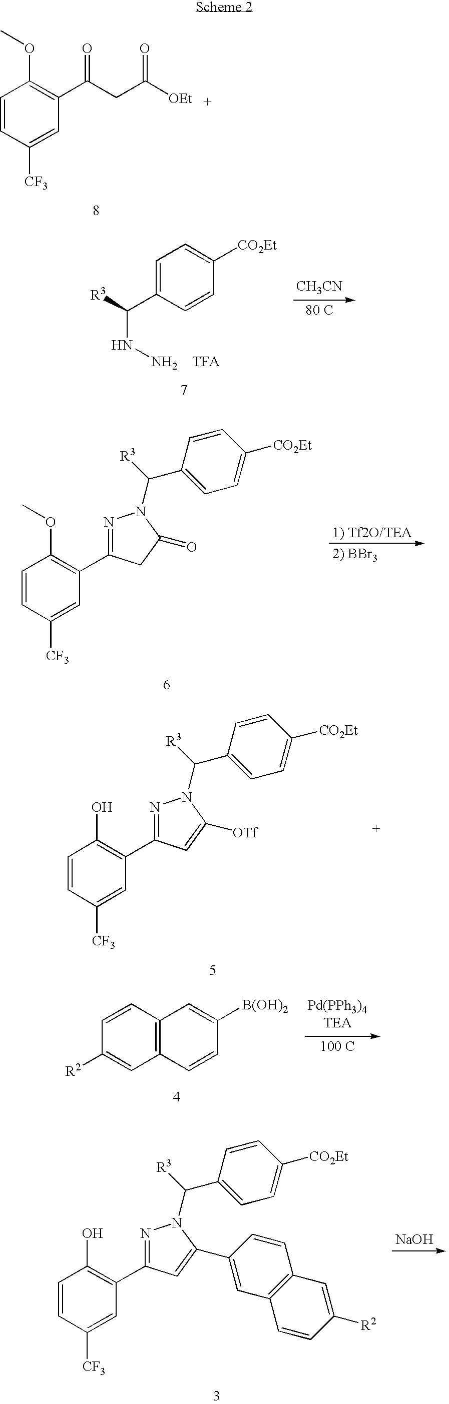

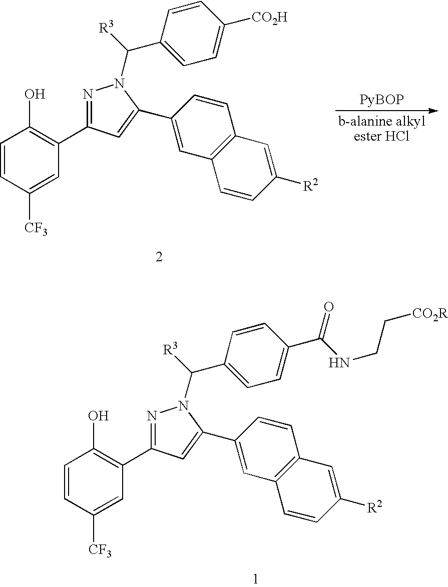

Compounds such as 8 may be conveniently prepared by a variety of methods familiar to those skilled in the art. One route is illustrated in Scheme 3 and described in Clay et al., Synthesis, 1993, 290. Acid chloride 9, which may be commercially available or readily prepared from the corresponding carboxylic acid by treatment with thionyl chloride at elevated temperatures or oxalyl chloride in a solvent such as methylene chloride in the presence of a catalytic amount of dimethylformamide (DMF) at room temperature, is treated with potassium ethyl malonate and magnesium chloride in the presence of base such as triethylamine in an aprotic solvent such as ethyl acetate for 1-16 h to give the beta-ketoester 8.

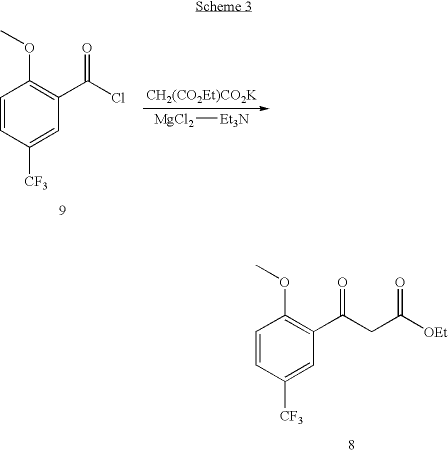

Benzyl hydrazine 7 may be prepared from the corresponding carbonyl analog 12 by condensation with tert-butylcarbazate in the presence of acetic acid in a nonpolar solvent such as toluene at elevated temperatures for 16 to 24 h, Scheme 4. The ketone 12 is commercially available or can be made from the palladium catalyzed coupling reaction of acid chloride 14 and arylzinc reagent 13 (see Negishi, et al., Tetrahedraon Letters, 1983, 5184). The acid chloride 14 is either commercially available or can be made from the corresponding carboxylic acid in refluxing thionyl chloride, or oxalyl chloride with or without solvent methylene chloride. The intermediate hydrazone 11 is then reduced with a hydride reducing agent such as sodium cyanoborohydride and 1 equivalent of p-toluenesulfonic acid, which should be added in a dropwise fashion. The reaction is carried out in a polar aprotic solvent such as tetrahydrofuran (THF) for 16-48 h at ambient temperature. Following aqueous work-up, the borane complex can be decomposed by slowly adding an aqueous solution of sodium hydroxide or other strong base to give carbamate 10 (see Calabretta et al., Synthesis, 1991, 536). Deprotection of the BOC group was effected by treatment with an acid such as trifluoroacetic acid in methylene chloride at ambient temperature for 0.25-2 h. The reaction can be performed with or without the addition of triisopropylsilane. The hydrazine 7 can either be used as its trifluoroacetate salt directly from the deprotection, or exchanged to hydrochloride salt by addition of aqueous hydrochloric acid and evaporation of the solvent. The protected hydrazine 10 can also be converted to 7 as a benzenesulfonic acid salt by heating a solution of 10 in alcohol with benzenesulfonic acid. Intermediate 10 contains a chiral center, the enantiomers can be resolved at this point by chromatography using a chiral stationary phase. Alternatively, hydrazone 11 can be directly reduced with hydrogen and a chiral catalyst such as a rhodium DuPHOS complex as described in Burk et al., Tetrahedron, 1994, 50, 4399, or a rhodium complex with {(R)-1-[(S)-2-diphenylphosphino)-ferrocenyl]ethyl(di-tert-butyl)phosphine (Hsiao, et al., JACS, 2004, 126, 9918). The solvent used for the reaction was generally an alcohol such as methanol or 2-propanol. The reduction can be carried out at elevated hydrogen pressure and at 20 to 80° C. ranges. This reaction would give material of enriched enantioselectivity which could be further purified by chiral chromatography as described above or by crystallization of the de-protected hydrazine salt to enhance the enantiomeric excess.

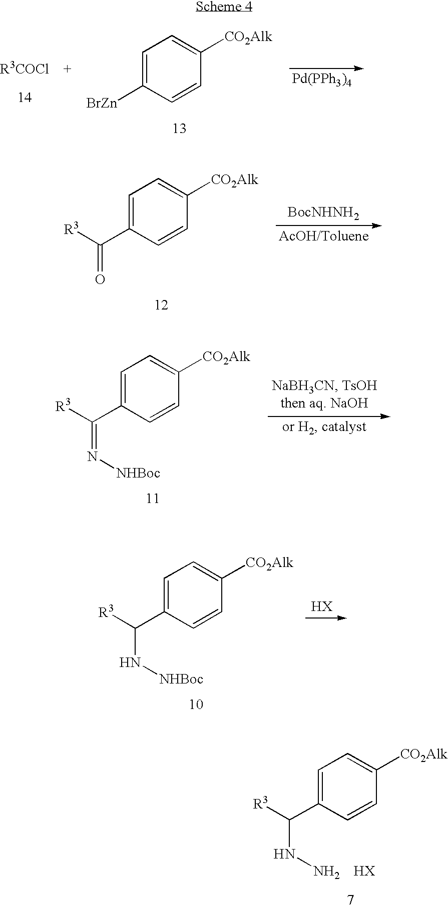

In another embodiment of the present invention, compound I can be synthesized via the route described in Scheme 5, where the R¹ group is introduced at an early stage of the synthesis. The R¹ group could be incorporated into 2-bromo-4-trifluoromethylphenol 22 by alkylation with alkyliodide (R¹I), or by Mitsunobu reaction with R¹OH or other means known to those skilled in the art, similar to the alkylation of compound 1 (vide supra). The resulting alkoxy phenyl bromide 22 can be converted to the benzoic acid 21, by means of metal halogen exchange with an alkyl Grignard reagent, generally isopropyl magnesium chloride, in an aprotic solvent such as THF over 12 h, followed by quenching the so formed arylmagnesium halide with dry ice. The resulting carboxylic acid 21 in turn may be converted to the beta-ketoester 19 in a similar process as described for compound 8. The beta-ketoester 19 can then be converted to the final compound I in similar reactions as described in Schemes 1 and 2, with the exception that de-methylation/alkylation steps should not be required. 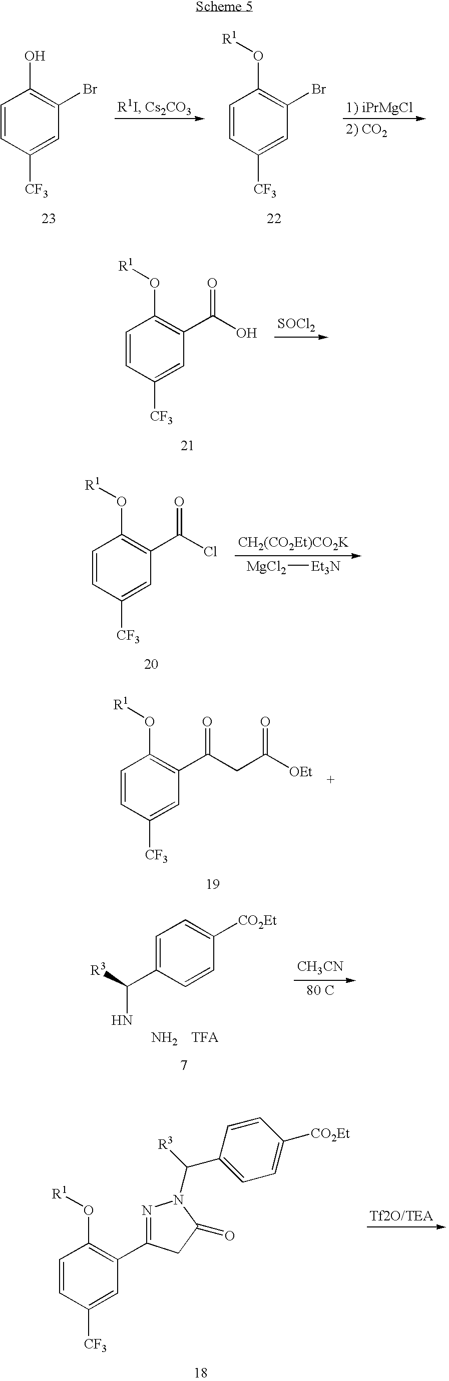 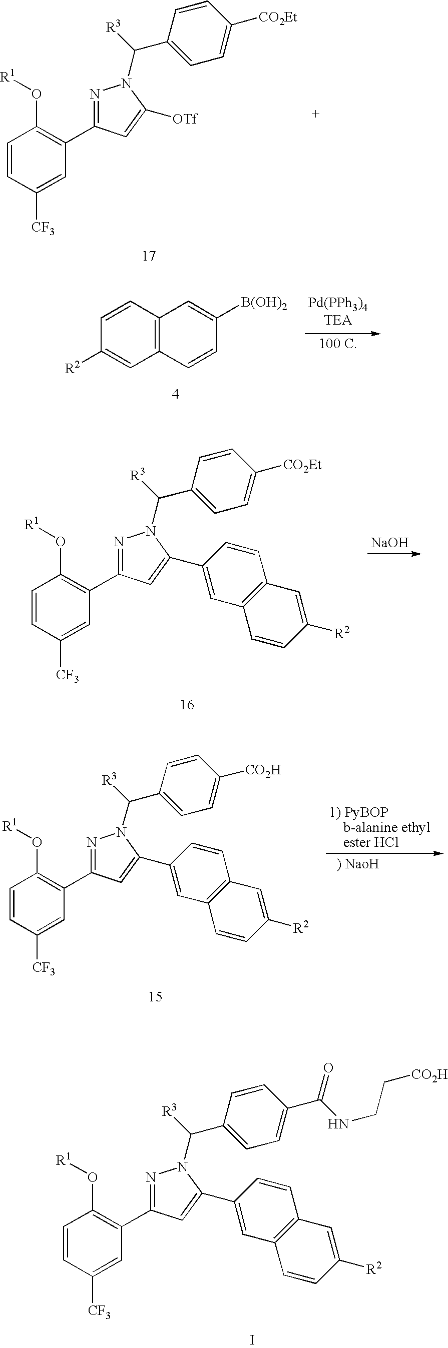
General experimental: Preparative HPLC was performed on a YMC-Pack Pro C18 column (150×20 mm i.d.) eluting at 20 mL/min with 0-100% acetonitrile in water (0.1% TFA). Flash column chromatography was carried out using Biotage system (pre packed silica gel columns).

The following examples are provided so that the invention might be more fully understood. They should not be construed as limiting the invention in any way. Preparation of intermediates is described below, these are used in the synthesis of Examples 1-13.

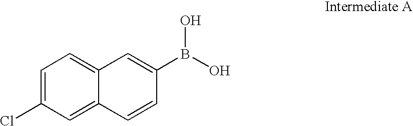

Step A 2-Bromo-6-chloronaphthalene. 6-Bromo-2-naphthoic acid (4.00 g, 15.9 mmol) was treated with 40 mL of thionyl chloride at 80° C. for 1 h. The mixture was concentrated in vacuo, and the resultant unpurified acid chloride (3 g, 11.1 mmol) was combined with 2,2′-azobisisobutyronitrile (731 mg, 4.45 mmol) in 25 mL of carbon tetrachloride and 15 mL of chlorobenzene. This mixture was added slowly via dropping funnel to a mixture of 2-mercaptopyridine-1-oxide sodium salt (1.99 g, 13.7 mmol) and 4-(dimethylamino)pyridine (150 mg, 1.23 mmol) at 100° C. After the addition was complete, the mixture was stirred for an additional 4 h, cooled, and the solid by-product precipitate removed by filtration. The filtrate was concentrated in vacuo and the residue purified by flash column chromatography (SiO₂, hexanes) to provide the title compound as a white solid. ¹H NMR (500 MHz, CDCl₃) δ: 8.02 (br s, 1H); 7.83 (d, J=1.6 Hz, 1H); 7.72 (d, J=8.7 Hz, 1H); 7.66 (d, J=8.7 Hz, 1H); 7.60 (dd, J=2.0, 8.9 Hz, 1H); 7.47 (dd, J=2.1, 8.7 Hz, 1H).

Step B 2-(6-Chloro-2-naphthyl)-4,4,5,5-tetramethyl-1,3,2-dioxaborolane. 2-Bromo-6-chloronaphthalene (205 mg, 0.849 mmol), bis(pinacolato)diboron (432 mg, 1.70 mmol), and potassium acetate (250 mg, 2.55 mmol) were dissolved in 12 mL of methyl sulfoxide. The mixture was de-oxygenated by four vacuum-nitrogen fill cycles, and dichloro[1,1′-bis(diphenylphosphino)ferrocene]palladium (II) dichloromethane adduct (70 mg, 0.085 mmol) was added. The resultant mixture was heated at 80° C. under a nitrogen atmosphere for 3 h then was allowed to sit at ambient temperature for 16 h. The mixture was diluted with ethyl acetate, and washed successively with two portions of water and one portion of brine. The organic layer was dried over magnesium sulfate, concentrated in vacuo, and the residue purified by flash column chromatography to provide the title compound. ¹H NMR (500 MHz, DMSO) δ: 8.34 (s, 1H); 8.10 (m, 2H); 7.89 (d, J=8.3 Hz, 1H), 7.76 (d, J=8.3 Hz, 1H); 7.54 (dd, J=1.8, 8.7 Hz, 1H); 1.33 (br s, 12H).

Step C (6-Chloro-2-naphthyl)boronic acid. 2-(6-Chloro-2-naphthyl)-4,4,5,5-tetramethyl-1,3,2-dioxaborolane (340 mg, 1.18 mmol) was suspended in a mixture of 20 mL of acetone and 5 mL of aqueous 2N hydrochloric acid and heated at 50° C. for 16 h. The product was purified by reverse phase HPLC to provide the title compound as a white powder. ¹H NMR (500 MHz, DMSO) δ: 8.38 (s, 1H); 8.23 (s, 2H); 8.01 (d, J=2.1 Hz, 1H); 7.95 (d, J=8.7 Hz, 1H); 7.91 (d, 8.2 Hz, 1H); 7.84 (d, J=8.2 Hz, 1H); 7.50 (dd, J=2.3, 8.7 Hz, 1H).

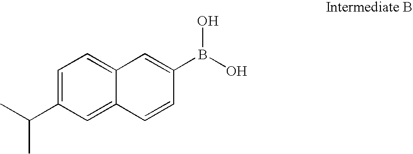

Step A 2-(6-Bromo-2-naphthyl)propan-2-ol. A solution of MeMgBr (3.0 M in ether, 14 ml, 42 mmol) was added to a solution of methyl 6-bromo-2-naphthoate (5 g, 19 mmol) in THF (50 ml) cooled in an ice-water bath. The reaction was stirred at room temperature for 1 hr before poured over to an ice-HCl mixture. This was extracted with ethyl acetate, washed with brine, and dried over Na₂SO₄. Evaporation of solvent left the title compound as a yellowish solid. ¹H NMR (500 MHz, CDCl₃): δ 1.60 (s, 6H); 7.52 (d, 1H); 7.66 (d, 1H); 7.76 (d, 2H); 7.93 (s, 1H); 8.00 (s, 1H).

Step B 2-Bromo-6 isopropenylnaphthalene. 2-(6-Bromo-2-naphthyl)propan-2-ol (3.0 g) was refluxed in HOAc (20 ml) for 1 h. After removing solvent at reduced pressure, the residue was purified by column chromatography over SiO₂, eluting with hexane, to give the title compound as a white powder. ¹H NMR (500 MHz, CDCl₃): δ 2.26 (s, 3H); 5.23 (s, 1H); 5.54 (s, 1H); 7.53 (d, 1H); 7.7 (m, 3H); 7.81 (s, 1H); 7.96 (s, 1H).

Step C (6 isopropenyl-2-naphthyl)boronic acid. A solution of BuLi (1.6 M in hexanes, 9.5 ml, 15 mmol) was added slowly to a mixture of 2-bromo-6-isopropenylnaphthalene (1 g, 4 mmol) and triisopropyl borate (5.2 ml, 22 mmol) in THF:Toluene (1:4, 20 ml) cooled at −78° C. After 10 min, the reaction was quenched with 2N HCl and extracted with ethyl acetate. The crude product was purified by reverse phase HPLC to give the title compound as a white powder. ¹H NMR (500 MHz, DMSO-d₆, 55° C.): δ 2.21 (s, 3H); 5.22 (s, 1H); 5.61 (s, 1H): 7.72 (d, 1H); 7.85 (m, 3H); 7.92 (s, 1H); 8.15 (br, 2H); 8.32 (s, 1H).

Step D (6 isopropyl-2-naphthyl)boronic acid. (6-Isopropenyl-2-naphthyl)boronic acid (300 mg), 5 mg of Pd/C, suspended in MeOH (5 ml), was stirred under hydrogen balloon for 1 h. After removing the catalyst by filtration, the crude product was purified by reverse phase HPLC to give the title compound as a brown solid. ¹H NMR (500 MHz, DMSO-d_{6,}): δ 1.30 (d, 6H); 3.05 (hepta, 1H); 7.42 (d, 1H); 7.67 (s, 1H); 7.77 (d, 1H); 7.82 (d, 2H); 7.98 (s, 2H); 8.30 (s, 1H).

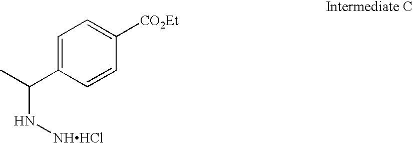

Step A tert-Butyl 2-{1-[4-(ethoxycarbonyl)phenyl]ethylidene}hydrazinecarboxylate. A solution of tert-butyl carbazate (13.90 g, 105 mmol) and ethyl 4-acetylbenzoate (20.00 g, 104 mmol) in toluene (120 mL) was stirred at 80° C. overnight (15 h). The title compound separated as crystalline solid and was collected by filtration of the mixture. HPLC/MS: m/z=307.3 (M+1)+, Rt=3.47 min. 1H NMR (500 MHz, CDCl₃): δ 8.05 (d, J=8.5 Hz, 2H), 7.88 (d, J=8.5 Hz, 2H), 7.79 (br s, 1H), 4.41 (q, J=7.0 Hz, 2H), 2.24 (s, 3H), 1.58 (s, 9H), 1.43 (t, J=7.0 Hz, 3H).

Step B-1 tert-Butyl 2-{1-[4-(ethoxycarbonyl)phenyl]ethyl}hvdrazinecarboxylate. In a N₂ filled round-bottomed flask equipped with serum caps and magnetic stirrer, NaBH₃CN (6.0 g, 0.095 mol) and tert-butyl-2-{1-[4-(ethoxycarbonyl)phenyl]ethylidene}hydrazine-carboxylate (25.6 g, 0.084 mol) were dissolved in THF (200 mL). A solution of p-toluenesulfonic acid monohydrate (17.3 g, 0.091 mol) in THF (50 mL) was added via syringe pump in about 10 h. The mixture was diluted with EtOAc (200 mL) and the suspension extracted with brine (150 mL). The organic phase was separated, dried (Na₂SO₄) and concentrated to give a white solid. The white solid was taken in CH₂Cl₂ (100 mL) and 1 N NaOH (100 mL) was added slowly. The suspension was stirred vigorously at r.t. for 1 h and then diluted with CH₂Cl₂ (100 mL). The organic phase was separated and washed with 1N HCl (2×150 mL), brine (2×150 mL), dried (Na₂SO₄) and concentrated to approximately 50 mL. The title compound precipitated as white solid and was collected by filtration and washed with dichloromethane. HPLC/MS: m/z=331.3 (M+Na)⁺. ¹H NMR (500 MHz, CDCl₃): δ 8.03 (d, J=8.0 Hz, 2H), 7.44 (d, J=8.0 Hz, 2H), 5.99 (br s, 1H), 4.40 (q, J=7.0 Hz, 2H), 4.29 (m, 1H), 1.45 (s, 9H), 1.41 (t, J=7.0 Hz, 3H), 1.35 (d, J=6.5 Hz, 3H).

Step B-2 (S)-tert-Butyl 2-{1-[4-(ethoxycarbonyl)phenyl]ethyl}hydrazinecarboxylate by asymmetric hydrogenation. In a glove box, MeOH (20 mL) was added to a mixture of {(R)-1-[(S)-2-diphenylphosphino)ferrocenyl]-ethyl(di-tert-butyl)phosphine (292 mg) and Rh(COD)₂BF₄ (199 mg) in a Fisher-Porter bottle fitted with a magnetic stir bar. The solution was aged 30-60 min, whereupon tert-butyl-2-{1-[4-(ethoxycarbonyl)phenyl]-ethylidene}hydrazinecarboxylate (10.0 g) and MeOH (80 mL) were added. The Fisher-Porter bottle was sealed, and transferred to a hydrogen manifold. After purging with hydrogen, the headspace was pressurized to 90 psig H₂ and the reaction heated to 50° C. After aging for 37 h, the reaction was discontinued. HPLC indicated 100% conversion to the title compound in 86% ee.

Step C {1-[4-(Ethoxycarbonyl)phenyl]ethyl}hydrazinium chloride. tert-Butyl 2-{1-[4-(ethoxycarbonyl)phenyl]ethyl}hydrazinecarboxylate (29 g, 94 mmol) was treated with 100 mL of TFA-DCM-triisopropylsilane (20:20:1) at room temperature for one hour. The mixture was concentrated under reduced pressure, and the residue was dissolved in water (100 mL), washed with DCM 2×. The DCM was back extracted with water 3×. HCl (5N, 20 mL) was added to the combined water solution and concentrated to ˜50 mL. CH₃CN (50 mL) was added and this was lyophilized to give the title compound as a white solid. NMR (500 MHz, acetone-d₆) δ: 1.34 (t, J=7.1 Hz, 3H); 1.67 (d, J=6.8 Hz, 3H); 4.33 (q, J=7.1 Hz, 2H), 4.97 (q, J=6.8 Hz, 1H), 7.76 (d, J=8.5 Hz, 2H), 7.97 (d, J=8.5 Hz, 2H). MS C₁₁H₁₆N₂O₂ Cald: 208.12; Obsd (M+1): 209.19.

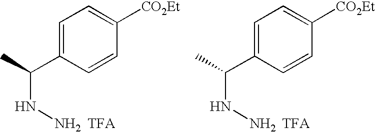

Step D {(1S)-1-[4-(ethoxycarbonyl)phenyl]ethyl}hydrazinium trifluoroacetate and {(1R)-1-[4-(ethoxycarbonyl)phenyl]ethyl}hydrazinium trifluoroacetate. tert-Butyl 2-{1-[4-(ethoxycarbonyl)phenyl]ethyl}hydrazinecarboxylate was analyzed by chiral HPLC using two sets of conditions. 1) Daicel column Chiralcel OJ, 40° C., 0.75 mL/min, 10% EtOH/90% n-heptane: t₁ 6.66 min; t₂ 12.25 min. Enantiomers were resolved on a preparative scale using this column (30% EtOH/70% n-Heptane). 2) Daicel column ChiralPak AD, 0.75 mL/min, 10% EtOH/90% n-heptane: t₁ 12.17 min; t₂15.49 min. Enantiomers were resolved on a preparative scale using this column (20% EtOH/80% n-Heptane). The fast moving enantiomer was identical in each case and was subsequently established to be the (S)-enantiomer ([α]_{D}²⁰=120° (c1.1, MeOH)), vide infra. The slower (R)-enantiomer was also isolated ([α]_{D}²⁰=+122° (c1.1, MeOH)).

Either enantiomer could be deprotected with 45:45:10 TFA:DCM:TIPS (40° C., 1.5 hr). The excess reagent and solvent was evaporated, and the residue was dissolved in water. The water solution was washed with DCM 2×. The DCM layers were back extracted with more water. The combined water solution was evaporated under vacuum (temp <45° C.), followed by azeotropic drying with toluene to give for the (S)-isomer-{(1S)-1-[4-(ethoxycarbonyl)phenyl]-ethyl}hydrazinium trifluoroacetate as a viscous oil. NMR (500 MHz, CD₃OD) δ: 1.38 (t, J=7.1 Hz, 3H); 1.49 (br d, J=7.0 Hz, 3H); 4.26 (br q, J=7.0 Hz, 1H); 4.37 (q, J=7.1 Hz, 2H); 7.54 (d, J=8.2 Hz, 2H); 8.07 (d, J=8.2 Hz, 2H). MS C₁₁H₁₆N₂O₂ Cald: 208.12; Obsd (M+1): 209.19. The other enantiomer {(1R)-1-[4-(ethoxycarbonyl)phenyl]ethyl}hydrazinium trifluoroacetate could be prepared in an identical fashion.

Determination of Absolute Configuration of Enantiomeric Hydrazines

Absolute configuration of the enantiomers of tert-butyl 2-{1-[4-(ethoxycarbonyl)phenyl]ethyl}hydrazinecarboxylate was established by conversion to ethyl 4-[1-(2-benzoylhydrazino)ethyl]benzoate, followed by comparison of the sign of optical rotation with that of the literature compound [Burk et al., Tetrahedron, 1994, 50, 4399-(S)-1-p-carboethoxyphenyl-1-(2-benzoylhydrazino)ethane (95% ee; [α]_{D}²⁰=−200.0° (c1, CHCl₃), HPLC Daicel Chiracel OJ, 40° C., 0.5 mL/min, 10% 2-propanol/90% hexane: Rₜ=33.1 min. (R)-isomer Rₜ=37.4 min.)].

Thus the slow moving enantiomer of tert-butyl 2-{1-[4-(ethoxycarbonyl)-phenyl]ethyl}hydrazinecarboxylate (0.40 g, 1.30 mmol) from a chiral separation as described above was treated with TFA/CH₂Cl₂ (1:1, 16 mL) for 1 h at r.t. The reaction was concentrated on a rotovap and the residual TFA was removed by co-evaporation from toluene. A portion of the resulting ethyl 4-(1-hydrazinoethyl)benzoate (0.36 mmol) was then dissolved in CH₂Cl₂ (5 mL) and cooled to −78° C. A solution of benzoyl chloride (56 μL, 0.48 mmol) and 2,6-di-tert-butyl-4-methylpyridine (74 mg, 0.36 mmol) in CH₂Cl₂ (1 mL) was added slowly at −78° C. After 3 h at −78° C., the reaction mixture was loaded quickly on a SiO₂ column and eluted with 30% EtOAc/hexane. Fractions containing product were concentrated and purified further on HPLC using Kromasil C₈ column (10% to 70% CH₃CN/H₂O/0.1% TFA, 12 min) to give (R)-(+)-ethyl 4-[1-(2-benzoylhydrazino)ethyl]benzoate. HPLC/MS: m/z=313.3 (M+1)⁺, Rₜ=3.12 min. Daicel column Chiralcel OJ, 40° C., 0.5 mL/min, 10% isopropanol/90% n-heptane: t 37.23 min; [α]_{D}²⁰=+110.6° (c1, CHCl₃); ¹H NMR (500 MHz, CDCl₃): δ 8.08 (2H, d, J=8.0 Hz), 7.82 (2H, d, J=7.5 Hz), 7.61 (1H, t, J=7.5 Hz), 7.54 (2H, d, J=8.0 Hz), 7.49 (2H, t, J=7.5 Hz), 4.62 (1H, q, J=7.0 Hz), 4.40 (2H, q, J=7.0 Hz), 1.73 (3H, d, J=6.5 Hz), 1.42 (3H, t, J=7.0 Hz); ¹³C NMR (500 MHz, CDCl₃): δ 167.43, 166.16, 141.05, 133.73, 131.91, 130.62, 129.88, 129.31, 128.16, 127.76, 61.85, 61.56, 17.44, 14.49.

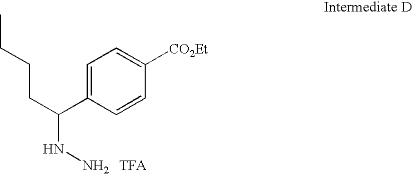

Step A Ethyl 4-pentanoylbenzoate. A solution of 4-ethoxycarbonylphenylzinc bromide (0.5 M in THF, 100 mL, 50 mmol) was added to a mixture of n-pentanoyl chloride (5.4 g, 45 mmol), and Pd(PPh₃)₄ (1 g, 2%) in THF (50 mL). The mixture was stirred at room temperature for 30 min under N₂. After removing solvent under reduced pressure, the residue was dissolved in ethyl acetate and washed with 1N HCl, brine, dried over Na₂SO₄. Flash column chromatography eluting with 5˜10% ethyl acetate in hexanes gave the title compound as a colorless oil. ¹H NMR (500 MHz, CDCl₃): δ 0.95 (t, 3H); 1.4 (m, 5H); 1.72 (pent, 2H); 2.99 (t, 2H); 4.21 (q, 2H); 7.99 (d, 2H); 8.12 (d, 2H). MS cald for C₁₄H₁₈O₃: 234.13; obsd: 235.28 (M+H).

Step B tert-butyl (2E)-2-{1-[4-(ethoxycarbonyl)phenyl]pentylidene}hydrazine-carboxylate. A solution of ethyl 4-pentanoylbenzoate (8 g, 34 mmol), t-butylcarbazide (5 g, 38 mmol), and HOAc (2.2 mL, ˜1 eq.) in dichloroethane (50 mL) was heated to 50° C. for 15 hr. After diluting with ethyl acetate, the mixture was washed with NaHCO₃ solution, brine, and dried over Na₂SO₄. Evaporation of solvent, after vacuum drying, gave the title compound as a white powder. ¹H NMR (500 MHz, CDCl₃): δ 0.95 (t, 3H); 1.40 (t, 3H); 1.44 (m, 2H); 1.54 (m, 2H); 1.57 (s, 9H); 2.62 (t, 2H); 4.38 (q, 2H); 7.84 (d, 2H); 8.03 (d, 2H).

Step C tert-butyl 2-{1-[4-(ethoxycarbonyl)phenyl]pentyl}hydrazinecarboxylate. Sodium cyanoborohydride (6 g, 95 mmol) was added to a solution of tert-butyl (2E)-2-{1-[4-(ethoxycarbonyl)phenyl]pentylidene}hydrazinecarboxylate (10.5 g, 30 mmol) in THF (200 mL) and MeOH (25 mL), followed by HOAc (2 mL). After stirring for 5 h, the mixture was diluted with ethyl acetate, washed with 5% K₂CO₃, brine, and dried over Na₂SO₄. Evaporation of solvent gave the title compound as colorless oil. ¹H NMR (500 MHz, DMSO-d₆): δ 0.79 (t, 3H); 1.06 (m, 1H); 1.15 (m, 1H); 1.21 (m, 2H); 1.31 (t, 3H); 1.33 (s, 9H); 1.42 (m, 1H); 1.66 (m, 1H); 4.3 (q, 2H); 4.62 (br, 1H); 7.42 (d, 2H); 7.88 (d, 2H); 8.0 (br, 1H). MS cald for C₁₉H₃₀N₂O₄: 350.22; obsd: 351.26 (M+H).

Step D Separation of enantiomers of tert-butyl 2-{1-[4-(ethoxycarbonyl)phenyl]-pentyl}hydrazinecarboxylate. The racemic compound was analyzed on ChiralPak AD (4.6×250 mm) column eluting with a linear gradient, from 3% to 50% in 20 min, of isopropyl alcohol in heptane at flow rate of 0.5 mL/min. The fast moving enantiomer has retention time of 13.9 min, and slow moving enantiomer of 17.2 min. The compound was separated on ChiralPak AD-H column with 35% isopropyl alcohol in heptane. The fast moving enantiomer has an optical rotation of [α]_{D}²⁰=+93° (c1.1, EtOH) and slow moving enantiomer [α]_{D}²⁰=94° (c1.1, EtOH).

Step E {1-[4-(ethoxycarbonyl)phenyl]pentyl}hydrazinium trifluoroacetate. tert-Butyl 2-{1-[4-(ethoxycarbonyl)-phenyl]pentyl}hydrazinecarboxylate was treated with 1:2 TFA:DCM at room temperature for 1 h. After removing excess reagent and solvent under reduced pressure, the residue was dissolved in toluene and evaporated (twice) to give an oil residue of the title compound. This hydrazine TFA salt decomposes at room temperature over time, and was always used without delay. ¹H NMR (500 MHz, CD₃OD): δ 8.08 (d, 2H); 7.52 (d, 2H); 4.38 (q, 2H); 4.06 (m, 1H); 1.93 (m, 1H); 1.72 (m, 1H); 1.39 (t, 3H); 1.20-1.40(m, 3H); 1.10 (m, 1H); 0.86 (t, 3H). MS cald for C₁₄H₂₂N₂O₂: 250.17; obsd: 251.18 (M+1H).

### EXAMPLE 1

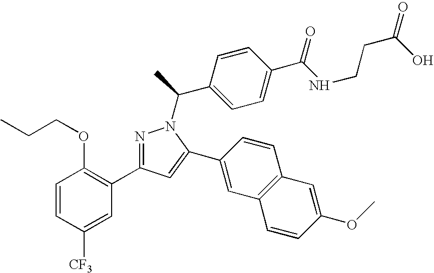

Step A 2-Methoxy-5-(trifluoromethyl)benzoic acid. A solution of 2-bromo-1-methoxy-4-(trifluoromethyl)-benzene (3 g, 11.2 mmol) in THF (5 mL) was added to a mixture of LiCl (0.5 g, 12 mmol) and isopropylmagnesium chloride (2 M in ether, 12 mL, 24 mmol) in THF (10 mL). After 15 h at room temperature the mixture was cooled to −78° C., and quenched with Dry Ice. The resulting mixture was allowed to warm up to room temperature and diluted with ethyl acetate, washed with 2N HCl. The ethyl acetate layer was then extracted with 5% K₂CO₃ three times. The combined base extracts was acidified and extracted with DCM twice. The DCM layer was then washed with brine, dried over Na₂SO₄. Evaporation of solvent yielded the title compound contaminated with isobutyric acid. ¹H-NMR (500 MHz, CDCl₃) δ: 4.12 (s, 3H); 7.16 (d, 1H); 7.81 (d, 1H); 8.41 (s, 1H).

Step B Ethyl 3-[2-methoxy-5-(trifluoromethyl)phenyl]-3-oxopropanoate. 2-Methoxy-5-(trifluoromethyl)benzoic acid (2.11 g) was refluxed in thionyl chloride (5 mL) for 2 hr, the excess reagent was evaporated off and vacuum dried to constant weight. This residue was dissolved in dry ethyl acetate (20 mL) and added slowly to a suspension which was prepared by mixing potassium ethyl malonate (1.85 g, 12 mMol), MgCl₂ (1.26 g, 14 mmol), and triethylamine (5.6 mL, 40 mmol) in dry ethyl acetate (30 mL) at 45° C. for 12 hr. The resulting mixture was stirred at room temperature overnight, then washed with 2N HCl, brine, and dried over Na₂SO₄. Flash column chromatography eluting with a gradient 0 to 10% ethyl acetate:hexane (1% HOAc) to give the title compound as a colorless oil. ¹H-NMR (500 MHz, CDCl₃) δ:1.22 (t, 3H); 3.96 (s, 3H); 3.97 (s, 2H); 4.18 (q, 2H); 7.07 (d, 1H); 7.75 (d, 1H); 8.16 (s, 1H); This compound in CDCl₃ also exists in the enol form in a ratio of 1:8 enol:keto form.

Step C Ethyl 4-((1S)-1-{3-[2-methoxy-5-(trifluoromethyl)phenyl]-5-oxo-4,5-dihydro-1H-pyrazol-1-yl}ethyl)benzoate. Ethyl 3-[2-methoxy-5-(trifluoromethyl)phenyl]-3-oxo-propanoate (2.94 g, 10.1 mmol) was heated with freshly prepared {(1S)-1-[4-(ethoxycarbonyl)phenyl]-ethyl}hydrazinium trifluoroacetate (3.2 g, 10.4 mmol) in acetonitrile (70 mL) for 10 hr. After removing solvent, the residue was purified by flash column chromatography eluting with 1:2 ethyl acetate:hexane to give the title compound as an oil. ¹H-NMR (500 MHz, CDCl₃) δ:1.37 (t, 3H); 1.80 (d, 3H): 3.80 (d, J=24 Hz), 1H); 3.84 (d, J=24 Hz, 1H); 3.91 (s, 3H); 4.36 (q, 2H); 5.58 (q, 1H); 7.01 (d, 1H); 7.51 (d, 2H); 7.62 (d, 1H); 8.02 (d, 2H); 8.19 (s, 1H). MS cald for C₂₂H₂₁F₃N₂O₄: 434.15; obsd: 435.21 (M+H).

Step D Ethyl 4-[(1S)-1-(3-[2-methoxy-5-(trifluoromethyl)phenyl]-5-{[(trifluoromethyl)sulfonyl]oxy}-1H-pyrazol-1-yl)ethyl]benzoate. Triflic anhydride (2.3 mL, 13.7 mmol) was added to a solution of ethyl 4-((1S)-1-{3-[2-methoxy-5-(trifluoromethyl)phenyl]-5-oxo-4,5-dihydro-1H-pyrazol-1-yl}ethyl)benzoate (2.96 g, 6.81 mmol), triethylamine (3.9 mL, 28 mmol) in DCM (70 mL) cooled at −78° C. The reaction was quenched with 2N HCl after 30 min. The reaction mixture was partitioned between ethyl acetate and 1N HCl. The organic layer was washed again with 1N HCl, and brine. The crude product was purified by column chromatography through SiO₂ column eluting with 5% to 7% ethyl acetate in hexanes to give the title compound as a colorless oil. ¹H-NMR (500 MHz, CDCl₃) δ:1.38 (t, 3H); 2.00 (d, 3H): 3.95 (s, 3H); 4.36 (q, 2H); 5.56 (q, 1H); 6.72 (s, 1H); 7.04 (d, 1H); 7.37 (d, 2H); 7.58 (d, 1H); 8.02 (d, 2H); 8.32 (s, 1H). MS cald for C₂₃H₂OF₆N₂O₆S: 566.09; obsd: 567.12 (M+H).

Step E Ethyl 4-[(1S)-1-(3-[2-hydroxy-5-(trifluoromethyl)phenyl]-5-{[(trifluoromethyl)-sulfonyl]oxy}-1H-pyrazol-1-yl)ethyl]benzoate. A solution of BBr₃ (1.0 M, 6.5 mL) was added to a solution of ethyl 4-[(1s)-1-(3-[2-methoxy-5-(trifluoromethyl)phenyl]-5-{[(trifluoromethyl)-sulfonyl]oxy}-1H-pyrazol-1-yl)ethyl]-benzoate (3.02 g, 5.33 mmol) in DCM (20 mL). After 2 hr, the reaction was diluted with ethyl acetate, washed with water twice, brine, and dried over Na₂SO₄. Evaporation of solvent yielded the title compound as a crystalline solid. ¹H-NMR (500 MHz, CDCl₃) δ:1.38 (t, 3H); 1.99 (d, 3H); 4.37 (q, 2H); 5.61 (q, 1H); 6.59 (s, 1H); 7.10 (d, 1H); 7.32 (d, 2H); 7.50 (d, 1H); 7.61 (s, 1H); 8.04 (d, 2H); 10.68 (s, 1H). MS cald for C₂₂H₁₈F₆N₂O₆S: 552.08; obsd: 553.22 (M+H).

Step F Ethyl 4-{(1S)-1-[3-[2-hydroxy-5-(trifluoromethyl)phenyl]-5-(6-methoxy-2-naphthyl)-1H-pyrazol-1-yl]ethyl}benzoate. Ethyl 4-[(1S)-1-(3-[2-hydroxy-5-(trifluoromethyl)-phenyl]-5-{[(trifluoromethyl)sulfonyl]oxy}-1H-pyrazol-1-yl)ethyl]benzoate (1.7 g, 3.08 mmol), (6-methoxy-2-naphthyl)boronic acid (0.75 g, 3.7 mmol), triethylamine (0.86 mL, 6.2 mmol), and Pd(PPh₃)₄ (0.28 g, 8% mol) were heated in toluene (50 mL) at 100° C. for 30 min. The reaction mixture was diluted with ethyl acetate, washed with 2N HCl, brine, and dried over Na₂SO₄. Flash column chromatography through SiO₂, eluting with a gradient of 10% to 33% ethyl acetate in hexane, afforded the title compound as a white powder. ¹H-NMR (500 MHz, CDCl₃) δ:1.39 (t, 3H); 1.96 (d, 3H); 2.18 (s, 11H); 3.96 (s, 3H); 4.38 (q, 2H); 5.65 (q, 1H); 6.82 (s, 1H); 7.11 (d, 1H); 7.18 (s, 1H); 7.21 (d, 2H); 7.22 (d, 1H); 7.33 (d, 1H); 7.48 (d, 1H); 7.68 (s, 11H); 7.69 (d, 1H); 7.80 (d, 1H); 7.86 (s, 1H); 7.99 (d, 2H). MS Cald for C₃₂H₂₇F₃N₂O₄ 560.19; Obsd: 561.25 (M+H).

Step G 4-{(1S)-[3-[2-Hydroxy-5-(trifluoromethyl)phenyl]-5-(6-methoxy-2-naphthyl)-1H-pyrazol-1-yl]ethyl}benzoic acid. A solution NaOH (1.5N, 3 mL) was added to a solution of ethyl 4-{(1S)-1-[3-[2-hydroxy-5-(trifluoromethyl)-phenyl]-5-(6-methoxy-2-naphthyl)-1H-pyrazol-1-yl]ethyl}-benzoate (0.6 g, 1.1 mmol) in 1:1 mixture of dioxane/methanol (12 mL). The reaction mixture was acidified after 3 hr, and product extracted with ethyl acetate, washed with brine and dried over Na₂SO₄. Evaporation of solvent afforded the title compound as a white solid. ¹H-NMR (500 MHz, CDCl₃) δ: 1.97 (d, 3H); 3.96 (s, 3H); 5.66 (q, 1H); 6.83 (s, 1H); 7.12 (d, 1H); 7.18 (s, 1H); 7.23 (d, 1H); 7.25 (d, 2H); 7.33 (d, 1H); 7.48 (d, 1H); 7.68 (s, 1H); 7.69 (d, 1H); 7.80 (d, 1H); 7.87 (s, 1H); 8.05 (d, 2H).

Step H tert-Butyl N-(4-{(1S)-1-[3-[2-hydroxy-5-(trifluoromethyl)phenyl]-5-(6-methoxy-2-naphthyl)-1H-pyrazol-1-yl]ethyl}benzoyl)-β-alaninate. A solution of PyBOP (340 mg, 0.65 mmol) in DMF (1.0 mL) was added to a mixture of 4-{(1S)-1-[3-[2-hydroxy-5-(trifluoromethyl)phenyl]-5-(6-methoxy-2-naphthyl)-1H-pyrazol-1-yl]ethyl}benzoic acid (290 mg, 0.54 mmol), β-alanine t-butyl ester hydrochloride (396 mg, 2.2 mmol), and DIEA (0.52 mL, 3 mmol) in DMF (3 mL). After 30 min, the reaction mixture was partitioned between ethyl acetate and 1N HCl. The organic layer was washed successively with 1N HCl two times, brine, and dried over Na₂SO₄. The crude product was purified with flash column chromatography eluting with 1:20 ethyl acetate: DCM to give the title compound as a dry foam. ¹H-NMR (500 MHz, DMSO-d₆) δ:1.35 (s, 9H); 1.92 (d, 3H); 2.42 (t, 2H); 3.40 (q, 2H); 3.90 (s, 3H); 5.82 (q, 1H); 7.13 (d, 1H); 7.19 (d, 2H); 7.23 (d, 1H); 7.27 (s, 1H); 7.40 (s, 1H); 7.48 (d, 1H); 7.54 (d, 1H); 7.72 (d, 2H); 7.85 (d, 1H); 7.92 (d, 1H); 7.93 (s, 1H); 8.19 (s, 1H); 8.44 (t, 1H). MS Cald for C₃₇H₃₆F₃N₃O₅: 659.26; Obsd: 660.29 (M+H).

Step I N-(4-{(1S)-1-[3-[2-Propoxy-5-(trifluoromethyl)phenyl]-5-(6-methoxy-2-naphthyl)-1H-pyrazol-1-yl]ethyl}benzoyl)-β-alanine. tert-Butyl N-(4-{(1S)-1-[3-[2-hydroxy-5-(trifluoromethyl)phenyl]-5-(6-methoxy-2-naphthyl)-1H-pyrazol-1-yl]ethyl}-benzoyl)-β-alaninate (4 mg) was stirred with propyliodide (0.02 mL, excess), Cs₂CO₃ (30 mg) in acetone (0.5 mL) for 2 hr. The solid was filtered off, and the residue was treated with 1:2 TFA/DCM (1 mL) for 1 hr. The excess reagent and solvent were removed, the crude product purified by reverse phase HPLC to give, after lyophilization, the title compound as a fluffy solid. ¹H-NMR (500 MHz, DMSO-d₆) δ: 1.01 (t, 3H); 1.84 (hex, 2H); 1.91 (d, 3H); 2.47 (t, 2H); 3.40 (q, 2H); 3.89 (s, 3H); 4.15 (t, 2H); 5.76 (q, 1H); 6.99 (s, 1H); 7.18 (d, 2H); 7.22 (d, 1H); 7.32 (d, 1H); 7.38 (s, 1H); 7.40 (d, 1H); 7.66 (d, 1H); 7.73 (d, 2H); 7.83 (d, 1H); 7.84 (s, 1H); 7.90 (d, 11H); 8.26 (s, 1H); 8.44 (t, 11H). MS Cald for C₃₆H₃₄F₃N₃O₅: 645.25; Obsd: 646.25 (M+H).

Example 2-5 were synthesized according to the procedures set forth in Example 1.

### EXAMPLE 2

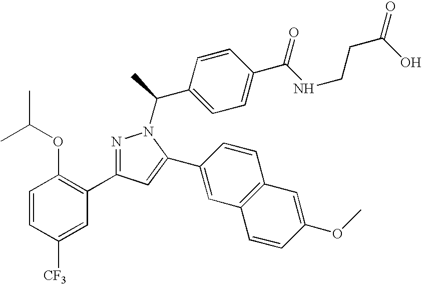

N-(4-{(1S)-1-[3-[2-isopropoxy-5-(trifluoromethyl)phenyl]-5-(6-methoxy-2-naphthyl)-1H-pyrazol-1-yl]ethyl}benzoyl)-β-alanine. ¹H-NMR (500 MHz, DMSO-d₆) δ:1.36 (d, 6H); 1.91 (d, 3H); 2.47 (t, 2H); 3.40 (q, 2H); 3.90 (s, 3H); 4.88 (hept, 1H); 5.84 (q, 1H); 6.99 (s, 1H); 7.19 (d, 2H); 7.22 (d, 1H); 7.34 (d, 1H); 7.38 (s, 1H); 7.42 (d, 1H); 7.64 (d, 1H); 7.73 (d, 2H); 7.84 (d, 1H); 7.85 (s, 1H); 7.90 (d, 1H); 8.28 (s, 11H); 8.44 (t, 11H). MS Cald for C₃H₃₄F₃N₃O₅: 645.25; Obsd: 646.27 (M+H).

### EXAMPLE 3

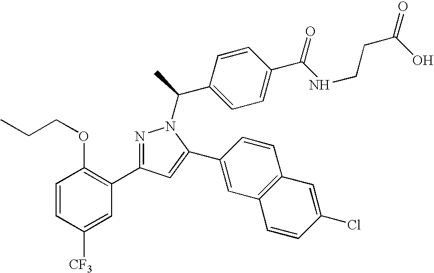

N-(4-{(1S)-1-[3-[2-Propoxy-5-(trifluoromethyl)phenyl]-5-(6-chloro-2-naphthyl)-1H-pyrazol-1-yl]ethyl}benzoyl)-β-alanine. MS Cald for C₃₅H₃₁ClF₃N₃O₄: 649.20; Obsd: 650.32 (M+H).

### EXAMPLE 4

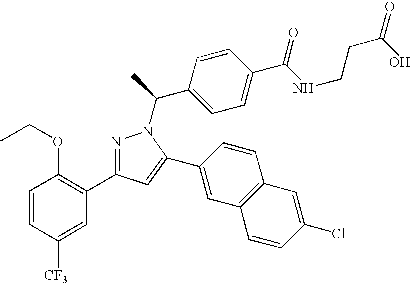

N-(4-{(1S)-[3-[2-Ethoxy-5-(trifluoromethyl)phenyl]-5-(6-chloro-2-naphthyl)-1H-pyrazol-1-yl]ethyl}benzoyl)-β-alanine. ¹H-NMR (500 MHz, DMSO-d₆) δ: 1.41 (t, 3H); 1.91 (d, 3H); 2.47 (t, 2H); 3.40 (q, 2H); 4.24 (q, 2H); 5.78 (q, 1H); 7.04 (s, 1H); 7.17 (d, 2H); 7.32 (d, 1H); 7.53 (d, 1H); 7.59 (d, 1H); 7.67 (d, 1H); 7.71 (d, 2H); 7.97 (d, 1H); 7.99 (s, 1H); 8.01 (d, 1H); 8.11 (s, 1H); 8.27 (s, 1H); 8.43 (t, 1H). MS Cald for C₃₄H₂₉ClF₃N₃O₄: 635.18; Obsd: 636.5.

### EXAMPLE 5

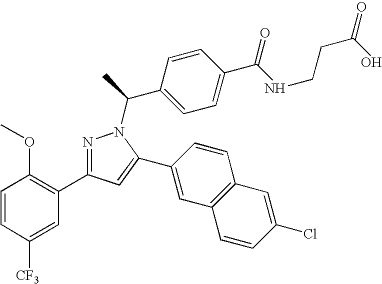

N-(4-{(1S)-1-[3-[2-Methoxy-5-(trifluoromethyl)phenyl]-5-(6-chloro-2-naphthyl)-1H-pyrazol-1-yl]ethyl}benzoyl)-β-alanine. ¹H-NMR (500 MHz, DMSO-ds) δ: 1.91 (d, 3H); 2.47 (t, 2H); 3.40 (q, 2H); 3.97 (s, 3H); 5.78 (q, 1H); 7.04 (s, 1H); 7.17 (d, 2H); 7.33 (d, 1H); 7.54 (d, 1H); 7.58 (d, 1H); 7.7 (m, 3H); 8.0 (m, 3H); 8.11 (s, 1H); 8.26 (s, 1H); 8.43 (t, 1H). MS Cald for C₃₃H₂₇ClF₃N₃O₄: 621.16; Obsd: 622.3.

### EXAMPLE 6

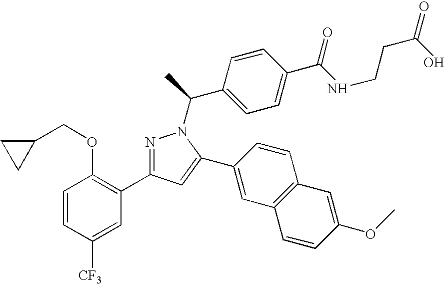

N-(4-{(1S)-1-[3-[2-(cyclopropylmethoxy)-5-(trifuoromethyl)phenyl]-5-(6-methoxy-2-naphthyl)-1H-pyrazol-1-yl]ethyl}benzoyl)-β-alanine. A solution of tert-Butyl N-(4-{(1S)-1-[3-[2-hydroxy-5-(trifluoromethyl)phenyl]-5-(6-methoxy-2-naphthyl)-1H-pyrazol-1-yl]ethyl}benzoyl)-β-alaninate (20 mg, 0.03 mmol), TMAD (9 mg, 0.15 mmol), cyclopropylmethyl alcohol (0.013 mL, 0.15 mol) in toluene (0.3 mLl) in a test tube was de-oxygenated by vacuum/N₂-fill cycles. A solution of Bu₃P (0.04 mL, 0.15 mmol) in toluene (0.18 mL) was then added. The reaction was stirred under N₂ atmosphere overnight. The crude product was purified by reverse phase HPLC to give an oil residue. This was then treated with 1:2 TFA:DCM (1 mL) for 30 min to give, after lyophilization, 16 mg of the title compound as a fluffy solid. ¹H-NMR (500 MHz, DMSO-d₆) δ: 0.39 (m, 2H); 0.58 (m, 2H); 1.34 (m, 1H); 1.91 (d, 3H); 2.47 (t, 2H); 3.41 (q, 2H); 3.89 (s, 3H); 4.05 (m, 2H); 5.78 (q, 1H); 7.11 (s, 1H); 7.19 (d, 2H); 7.22 (d, 1H); 7.28 (d, 1H); 7.38 (s, 1H); 7.42 (d, 1H); 7.64 (d, 1H); 7.73 (d, 2H); 7.83 (d, 1H); 7.85 (s, 1H); 7.90 (d, 1H); 8.28 (s, 11H); 8.44 (t, 1H). MS Cald for C₃₇H₃₄F₃N₃O₅: 657.25; Obsd: 658.30 (M+H).

Example 7 and 8 were synthesized in accordance with the procedures set forth in Example 6.

### EXAMPLE 7

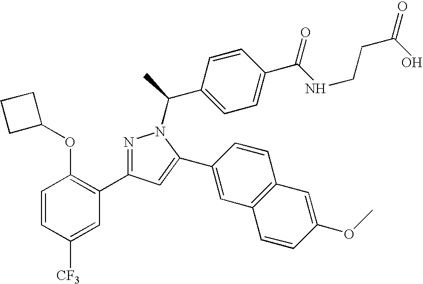

N-(4-{(1S)-1-[3-[2-(cyclobutyloxy)-5-(trifuoromethyl)phenyl]-5-(6-methoxy-2-naphthyl)-1H-pyrazol-1-yl]ethyl}benzoyl)-β-alanine. ¹H-NMR (500 MHz, DMSO-d₆) δ: 1.67 (m, 1H); 1.82 (m, 1H); 1.91 (d, 3H); 2.14 (m, 2H); 2.47 (t, 2H); 2.5 (m, 2H); 3.40 (q, 2H); 3.89 (s, 3H); 4.92 (pent, 1H); 5.78 (q, 1H); 7.02 (s, 1H); 7.14 (d, 1H); 7.19 (d, 2H); 7.22 (d, 1H); 7.38 (s, 1H); 7.42 (d, 1H); 7.63 (d, 1H); 7.73 (d, 2H); 7.84 (d, 1H); 7.87 (s, 1H); 7.90 (d, 1H); 8.26 (s, 1H); 8.44 (t, 11H). MS Cald for C₃₇H₃₄F₃N₃O₅: 657.25; Obsd: 658.3 (M+H).

### EXAMPLE 8

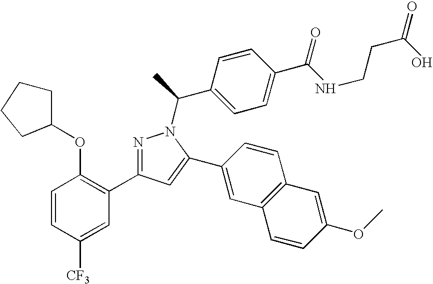

N-(4-{(1S)-1-[3-[2-(cyclopentyloxy)-5-(trifluoromethyl)phenyl]-5-(6-methoxy-2-naphthyl)-1H-pyrazol-1-yl]ethyl}benzoyl)-β-alanine. ¹H-NMR (500 MHz, DMSO-d₆) δ: 1.4 (m, 2H); 1.7 (m, 2H); 1.82 (m, 2H); 1.91 (d, 3H); 1.98 (m, 2H); 2.47 (t, 2H); 3.40 (q, 2H); 3.89 (s, 3H); 5.08 (m, 1H); 5.78 (q, 1H); 6.94 (s, 1H); 7.18 (d, 2H); 7.22 (d, 1H); 7.31 (d, 1H); 7.38 (s, 1H); 7.40 (d, 1H); 7.64 (d, 1H); 7.73 (d, 2H); 7.83 (d, 1H); 7.84 (s, 1H); 7.90 (d, 1H); 8.26 (s, 1H); 8.44 (t, 1H). MS Cald for C₃₈H₃₆F₃N₃O₅: 671.26; Obsd: 672.30 (M+H).

### EXAMPLE 9

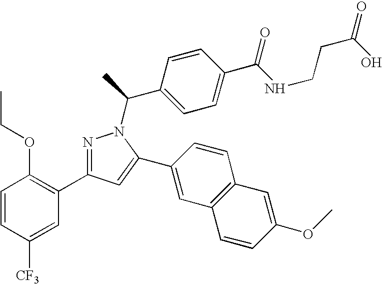

Step A 2-Bromo-1-ethoxy-4-(trifluoromethyl)benzene. To a solution of 2-bromo-4-trifluoromethylphenol (10 g, 42 mmol) in DMF (50 mL) was added Cs₂CO₃ (16.3 g, 50 mmol), and ethyliodide (10 mL, 100 mmol). The mixture was stirred at room temperature for 20 min, diluted with ethyl acetate (300 mL), and acidified with HCl (1N, 300 mL). The organic layer was washed with HCl (1N, 300 mL), brine (2×), and dried over Na₂SO₄. Evaporation of solvent afforded the title compound as an oil. NMR (500 MHz, CDCl₃) δ: 1.50 (t, 3H); 4.16 (q, 2H); 6.92 (d, 1H); 7.52, (d, 1H); 7.80 (s, 1H).

Step B 2-Ethoxy-5-(trifluoromethyl)benzoic acid. A solution of 2-bromo-1-ethoxy-4-(trifluoromethyl)benzene (11.2 g, 42 mmol) in THF (30 mL) was added to a mixture of iPrMgCl (2.0 M in ether, 50 mL, 100 mmol), LiCi (2 g, 48 mmol) in THF (50 μL) at room temperature. After 3 hr, the mixture was transferred to Dry Ice (excess). The resulting mixture was allowed to warm up to room temperature and was diluted with ethyl acetate (400 mL), washed with HCl (2N, 2×300 mL). The organic layer was extracted with 5% K₂CO₃ three times. The combined base extracts were acidified with concentrated HCl and extracted with DCM 2×. This DCM solution was washed with brine, dried over Na₂SO₄. Evaporation of solvent and vacuum drying yielded the title compound as a white solid. NMR (500 MHz, CDCl₃) δ:1.43 (t, 3H); 4.20 (q, 2H); 7.24 (d, 1H); 7.77 (d, 1H); 8.02 (s, 1H).

Step C Ethyl 3-[2-ethoxy-5-(trifluoromethyl)phenyl]-3-oxopropanoate. 2-Ethoxy-5-(trifluoromethyl)benzoic acid (6.7 g, 22.8 mmol) was refluxed in thionyl chloride (20 mL) for 2 hr. The excess reagent was evaporated and the residue vacuum dried to constant weight. This residue was dissolved in dry ethyl acetate (50 mL) and added slowly to a suspension which was prepared by mixing potassium ethyl malonate (5 g, 32.5 mmol), MgCl2 (3.6 g, 40 mmol), and triethylamine (14 mL, 100 mmol) in dry ethyl acetate (100 mL) at 45° C. for 12 hr. The resulting mixture was stirred at room temperature overnight. The reaction mixture was washed with 2N HCl, brine, and dried over Na₂SO₄. Flash column chromatograph eluting with a gradient of 0 to 10% ethyl acetate in hexane (1% HOAc) gave the title compound as colorless oil. ¹H-NMR (500 MHz, CDCl₃) δ:1.22 (t, 3H); 1.51 (t, 3H); 4.01 (s, 2H); 4.2 (m, 4H); 7.02 (d, 1H); 7.72 (d, 1H); 8.15 (s, 1H); This compound in CDCl₃ exists in the enol form at a ratio of 1:5 enol:keto form.

Step D Ethyl 4-[(1S)-1-(3-[2-ethoxy-5-(trifluoromethyl)phenyl]-5-{[(trifluoromethyl)sulfonyl]oxy}-1H-pyrazol-1-yl)ethyl]benzoate. Ethyl 3-[2-ethoxy-5-(trifluoromethyl)-phenyl]-3-oxopropanoate (5.41 g, 18.7 mmol), {(1S)-1-[4-(ethoxycarbonyl)phenyl]-ethyl}diazanium benzenesulfonate (7.32 g, 20 mmol) were heated in acetonitrile (50 mL) at 80° C. for 10 hr. The reaction mixture was then diluted with ethyl acetate, washed successively with HCl 2×, brine, and dried over Na₂SO₄. Evaporation of solvent and vacuum drying left a residue which was dissolved in DCM (100 mL) and cooled to −78° C. Triethylamine (10 mL, 71 mmol) and triflic anhydride (6 mL, 33 mmol) were added. The reaction was allowed to warm up to room temperature, washed with 2N HCl, brine, and dried over Na₂SO₄. Flash column chromatograph eluting with a gradient of 0 to 5% ethyl acetate in hexane afforded the title compound as a pale yellow oil. ¹H-NMR (500 MHz, CDCl₃) δ:1.37 (t, 3H); 1.49 (t, 3H); 2.00 (d, 3H); 4.18 (q, 2H); 4.37 (q, 2H); 5.58 (q, 1H); 6.81(s, 1H); 7.01 (d, 1H); 7.38 (d, 2H); 7.56 (d, 1H); 8.02 (d, 2H); 8.34 (s, 1H). MS Cald for C₂₄H₂₂F₆N₂O₆S 580.11; Obsd: 581.31 (M+H).

Step E Ethyl 4-{(1S)-1-[3-[2-ethoxy-5-(trifluoromethyl)phenyl]-5-(6-methoxy-2-naphthyl)-1H-pyrazol-1-yl]ethyl}benzoate. Ethyl 4-[(1S)-1-(3-[2-ethoxy-5-(trifluoromethyl)-phenyl]-5-{[(trifluoromethyl)sulfonyl]oxy}-1H-pyrazol-1-yl)ethyl]benzoate (6.03 g, 10.4 mmol), (6-methoxy-2-naphthyl)boronic acid (3.1 g, 15 mmol), triethylamine (4.2 mL, 30 mmol), and Pd(PPh₃)₄ (0.5 g, 4% mol) were heated in toluene (50 mL) at 100° C. for 30 min. The reaction mixture was diluted with ethyl acetate, washed with 2N HCl, brine, and dried over Na₂SO₄. Flash column chromatography eluting with a gradient of 0% to 10% ethyl acetate in hexane, afforded the title compound as a white powder. ¹H-NMR (500 MHz, CDCl₃) δ:1.38 (t, 3H); 1.51 (t, 3H); 1.98 (d, 3H); 3.96 (s, 3H); 4.20 (q, 2H); 4.38 (q, 2H); 5.60 (q, 1H); 6.98 (s, 1H); 7.04 (d, 1H); 7.15 (s, 1H); 7.19 (d, 1H); 7.30 (d, 2H); 7.31 (d, 1H); 7.52 (d, 1H); 7.64 (s, 11H); 7.68 (d, 1H); 7.75 (d, 11H); 7.99 (d, 2H); 8.45 (s, 11H). MS Cald for C₃₄H₃₁F₃N₂O₄ 588.22; Obsd: 589.47 (M+H).

Step F 4-{(1S)-1-[3-[2-Ethoxy-5-(trifluoromethyl)phenyl]-5-(6-methoxy-2-naphthyl)-1H-pyrazol-1-yl]ethyl}benzoic acid. Ethyl 4-{(1S)-1-[3-[2-ethoxy-5-(trifluoromethyl)-phenyl]-5-(6-methoxy-2-naphthyl)-1H-pyrazol-1-yl]ethyl}benzoate (5.1 g, 8.7 mmol) was dissolved in 70 mL of dioxane:methanol (5:2). A solution of NaOH (1.5N, 10 mL) was added. The mixture was heated to 60° C. for 30 min. The cooled reaction mixture was acidified with HCl, extracted with DCM. The DCM solution was concentrated to give the title compound as a white solid. ¹H-NMR (500 MHz, acetone-d₆) δ:1.49 (t, 3H); 2.01 (d, 3H); 3.93 (s, 3H); 4.29 (q, 2H); 5.85 (q, 11H); 7.10 (s, 11H); 7.21 (d, 11H); 7.28 (d, 1H); 7.35 (s, 1H); 7.37 (d, 2H); 7.42 (d, 1H); 7.63 (d, 1H); 7.79 (s, 1H); 7.82 (d, 1H); 7.88 (d, 1H); 7.98 (d, 2H); 8.50 (s, 1H).

Step G Ethyl N-(4-{(1S)-1-[3-[2-ethoxy-5-(trifluoromethyl)phenyl]-5-(6-methoxy-2-naphthyl)-1H-pyrazol-1-yl]ethyl}benzoyl)-β-alaninate. A solution of PyBOP (6.76 g, 13 mmol) in DMF (20 mL) was added slowly to a mixture of 4-{(1S)-1-[3-[2-Ethoxy-5-(trifluoromethyl)phenyl]-5-(6-methoxy-2-naphthyl)-1H-pyrazol-1-yl]ethyl}benzoic acid (4.9 g, 8.7 mmol), β-alanine ethyl ester hydrochloride (3.07 g, 20 mmol), Et₃N (5.6 mL, 40 mmol), DMAP (0.5 g) in DMF (30 mL). After 30 min, the reaction mixture was diluted with ethyl acetate (300 mL), washed successively with 1N HCl (2×), brine, and dried over Na₂SO₄. Flash column chromatography, eluting with a gradient of 20%-45% ethyl acetate in hexane, gave the title compound as a white solid. ¹H-NMR (500 MHz, CDCl₃) δ:1.26 (t, 3H); 1.51 (t, 3H); 1.96 (d, 3H); 2.62 (t, 2H); 3.71 (q, 2H); 3.95 (s, 3H); 4.15 (q, 2H); 4.18 (q, 2H); 5.59 (q, 1H); 6.83 (t, 1H); 6.98 (s, 1H); 7.02 (d, 1H); 7.16 (s, 1H); 7.19 (d, 1H); 7.30 (d, 2H); 7.31 (d, 1H); 7.52 (d, 1H); 7.65 (s, 1H); 7.69 (d, 1H); 7.70 (d, 2H); 7.75 (d, 11H); 8.46 (s, 11H). MS Cald for C₃₇H₃₆F₃N₃O₅ 659.26; Obsd: 660.51 (M+H).

Step H N-(4-{(1S)-1-[3-[2-Ethoxy-5-(trifluoromethyl)phenyl]-5-(6-methoxy-2-naphthyl)-1H-pyrazol-1-yl]ethyl}benzoyl)-β-alanine. A solution of NaOH (1.5N, 10 mL) was added slowly to a solution of ethyl N-(4-{(1s)-1-[3-[2-ethoxy-5-(trifluoromethyl)phenyl]-5-(6-methoxy-2-naphthyl)-1H-pyrazol-1-yl]ethyl}benzoyl)-β-alaninate (5.54 g, 8.4 mmol) in THF:MeOH (1:1, 200 mL). The mixture was stirred at room temperature for 30 min before acidified with HCl, extracted with ethyl acetate. Evaporation of ethyl acetate left a glassy residue, which was dissolved in acetonitrile:water (4:1, 100 mL) and lyophilized to give N-(4-{(1S)-1-[3-[2-ethoxy-5-(trifluoromethyl)phenyl]-5-(6-methoxy-2-naphthyl)-1H-pyrazol-1-yl]ethyl}benzoyl)-β-alanine as a fluffy powder. ¹H-NMR (500 MHz, DMSO-d₆) δ: 1.42 (t, 3H); 1.91 (d, 3H); 2.47 (t, 2H); 3.40 (q, 2H); 3.89 (s, 3H); 4.24 (q, 2H); 5.76 (q, 1H); 7.00 (s, 1H); 7.19 (d, 2H); 7.22 (d, 1H); 7.31 (d, 1H); 7.38 (s, 1H); 7.42 (d, 1H); 7.66 (d, 1H); 7.73 (d, 2H); 7.84 (d, 1H); 7.85 (s, 1H); 7.90 (d, 1H); 8.27 (s, 1H); 8.44 (t, 1H). MS Cald for C₃₅H₃₂F₃N₃O₅: 631.23; Obsd: 632.23 (M+H). Specific rotation: [α]_{D}²⁰=−27° (c1.1, EtOH).

The enantiomeric compound, N-(4-{(1R)-1-[3-[2-Ethoxy-5-(trifluoromethyl)phenyl]-5-(6-methoxy-2-naphthyl)-1H-pyrazol-1-yl]ethyl}benzoyl)-β-alanine, was prepared in a similar manner. Specific rotation: [α]_{D}²⁰=+24° (c1.1, EtOH).

Examples 10-13 were synthesized following the same steps described in Example 1 and 9.

### EXAMPLE 10

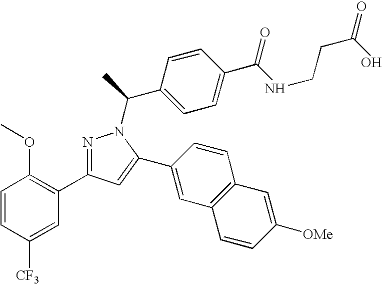

N-(4-{(1S)-1-[3-[2-methoxy-5-(trifluoromethyl)phenyl]-5-(6-methoxy-2-naphthyl)-1H-pyrazol-1-yl]ethyl}benzoyl)-β-alanine. ¹H-NMR (500 MHz, DMSO-46) δ: 1.91 (d, 3H); 2.47 (t, 2H); 3.40 (q, 2H); 3.89 (s, 3H); 3.99 (s, 3H); 5.78 (q, 1H); 7.10 (s, 1H); 7.19 (d, 2H); 7.22 (d, 1H); 7.34 (d, 1H); 7.38 (s, 1H); 7.42 (d, 1H); 7.69 (d, 1H); 7.73 (d, 2H); 7.84 (d, 1H); 7.88 (s, 1H); 7.90 (d, 1H); 8.26 (s, 1H); 8.44 (t, 11H). MS Cald for C₃₄H₃₀F₃N₃O₅: 617.21; Obsd: 618.22 (M+H).

### EXAMPLE 11

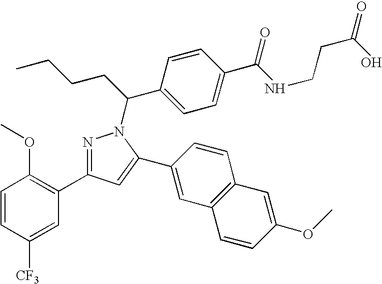

N-[4-((1S)-{5-(6-methoxy-2-naphthyl)-3-[2-methoxy-5-(trifluoromethyl)phenyl]-1H-pyrazol-1-yl}pentyl)benzoyl]-β-alanine. This compound was synthesized using the slow moving enantiomer of tert-butyl 2-{1-[4-(ethoxycarbonyl)phenyl]pentyl}-hydrazinecarboxylate. ¹H-NMR (500 MHz, acetone-d₆) δ: 8.50 (s, 1H), 7.91 (d, 1H), 7.85 (d, 2H), 7.82(d, 1H), 7.79 (s, 1H), 7.66 (d, 1H); 7.49 (d, 2H); 7.40 (d, 1H); 7.38 (s, 1H); 7.32 (d, 1H); 7.21 (d, 1H); 7.02 (s, 1H); 5.52 (q, 1H), 4.04 (s, 3H), 3.95 (s, 3H), 3.62 (m, 2H), 2.65 (m, 1H), 2.64 (t, 2H), 2.16 (m, 1H), 1.17-1.30 (m, 4H), 0.81 (t, 3H). MS cald for C₃₇H₃₆F₃N₃O₅: 659.26. obsd: 660.34 (M+H).

### EXAMPLE 12

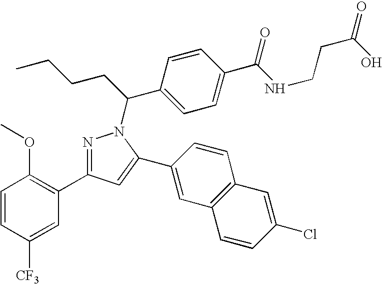

N-[4-((1S)-1-{5-(6-chloro-2-naphthyl)-3-[2-methoxy-5-(trifluoromethyl)phenyl]-1H-pyrazol-1-yl}pentyl)benzoyl]-β-alanine. This compound was synthesized using the slow moving enantiomer of tert-butyl 2-{1-[4-(ethoxycarbonyl)phenyl]pentyl}-hydrazinecarboxylate. ¹H-NMR (500 MHz, acetone-d₆) δ: 8.50 (s, 1H), 8.05 (s, 1H), 8.01 (d, 1H), 7.96 (d, 1H), 7.92 (s, 1H), 7.85(d, 2H), 7.67 (d, 1H), 7.57 (d, 1H), 7.53 (d, 1H), 7.49(d, 2H), 7.33 (d, 1H), 7.08 (s, 1H), 5.53 (q, 1H), 4.05 (s, 3H), 3.62 (t, 2H), 2.66 (m, 1H), 2.64 (t, 2H), 2.16 (m, 11H), 1.18-1.30 (m, 4H), 0.81 (t, 3H). MS Cald for C₃₆H₃₃ClF₃N₃O₄: 663.21; Obsd: 664.31 (M+H).

### EXAMPLE 13

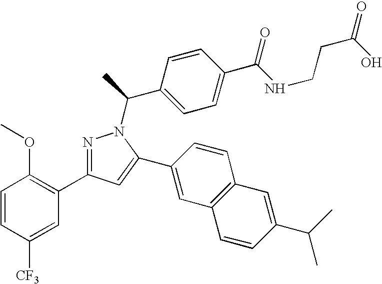

N-[4-((1S)-1-{5-(6 isopropyl-2-naphthyl)-3-[2-methoxy-5-(trifluoromethyl)phenyl]-1H-pyrazol-1-yl}ethyl)benzoyl]-β-alanine. ¹H-NMR (500 MHz, acetone-d₆) δ: 1.34 (d, 6H); 1.98 (d, 3H); 2.62 (t, 2H); 3.11 (hept, 1H); 3.61 (m, 2H); 4.04 (s, 3H); 5.82 (q, 1H); 7.06 (s, 1H); 7.32 (m, 3H); 7.45 (d, 1H); 7.52 (d, 1H); 7.65 (d, 1H); 7.79 (d, 1H); 7.80 (d, 2H); 7.82 (d, 1H); 7.85 (s, 1H); 7.92 (d, 1H); 8.48 (s, 1H). MS Cald for C₃H₃₄F₃N₃O₄: 629.25; Obsd: 630.69 (M+H).

Biological Assays

The ability of the compounds of the present invention to inhibit the binding of glucagon and their utility in treating or preventing type 2 diabetes mellitus and the related conditions can be demonstrated by the following in vitro assays. Glucagon Receptor Binding Assay

A stable CHO (Chinese hamster ovary) cell line expressing cloned human glucagon receptor was maintained as described (Chicchi et al. J Biol Chem 272, 7765-9(1997); Cascieri et al. J Biol Chem 274, 8694-7(1999)). To determine antagonistic binding affinity of compounds 0.002 mg of cell membranes from these cells were incubated with ¹²⁵I-Glucagon (New England Nuclear, MA) in a buffer containing 50mM Tris-HCl (pH 7.5), 5 mM MgCl, 2mM EDTA, 12% Glycerol, and 0.200 mg WGA coated PVT SPA beads (Amersham), +/− compounds or 0.001 MM unlabeled glucagon. After 4-12 hours incubation at room temperature, the radioactivity bound to the cell membranes was determined in a radioactive emission detection counter (Wallac-Microbeta). Data was analyzed using the software program Prism from GraphPad. The IC₅₀ values were calculated using non-linear regression analysis assuming single site competition. IC₅₀ values for the compounds of the invention are generally in the range of as low as about 1 mM to as high as about 10 nM, and thus have utility as glucagon antagonists.

Inhibition of Glucagon-Stimulated Intracellular cAMP Formation

Exponentially growing CHO cells expressing human glucagon receptor were harvested with the aid of enzyme-free dissociation media (Specialty Media), pelleted at low speed, and re-suspended in the Cell Stimulation Buffer included in the Flash Plate cAMP kit (New England Nuclear, SMP0004A). The adenylate cyclase assay was setup as per manufacturer instructions. Briefly, compounds were diluted from stocks in DMSO and added to cells at a final DMSO concentration of 5%. Cells prepared as above were preincubated in flash plates coated with anti-cAMP antibodies (NEN) in presence of compounds or DMSO controls for 30 minutes, and then stimulated with glucagon (250 pM) for an additional 30 minutes. The cell stimulation was stopped by addition of equal amount of a detection buffer containing lysis buffer as well as ¹²⁵I-labeled cAMP tracer (NEN). After 3 hours of incubation at room temperature the bound radioactivity was determined in a liquid scintillation counter (TopCount-Packard Instruments). Basal activity (100% inhibition) was determined using the DMSO control while 0% inhibition was defined at the amount of pmol cAMP produced by 250 pM glucagon. Generally the compounds of the present invention inhibit cAMP formation at a concentration less than about 50 nM, and in select cases, less than about 5 nM.

Additionally, the compounds of the present invention demonstrate desirable pharmacodynamic and pharmacokinetic properties.

Certain embodiments of the invention has been described in detail; however, numerous other embodiments are contemplated as falling within the invention. Thus, the claims are not limited to the specific embodiments described herein. All patents, patent applications and publications that are cited herein are hereby incorporated by reference in their entirety.

What is claimed is:

1. A compound of formula I: 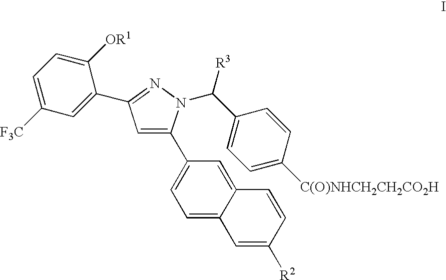
or a pharmaceutically acceptable salt or solvate thereof wherein:
R¹ represents C₁₋₆alkyl;
R² represents halo, C₁₋₃alkyl or OC₁₋₃alkyl and
R³ represents C₁₋₆alkyl.

2. A compound in accordance with claim 1 wherein: R¹ is selected from the group consisting of: methyl, ethyl, n-propyl, isopropyl, cyclopropyl, cyclopropylmethyl, cyclobutyl and cyclopentyl.

3. A compound in accordance with claim 1 wherein: R² is selected from the group consisting of: chloro, methyl, ethyl, propyl, isopropyl and methoxy.

4. A compound in accordance with claim 1 wherein: R³ is selected from the group consisting of: methyl, ethyl, n-propyl and n-butyl.

5. A compound in accordance with claim 1 wherein:
R¹ is selected from the group consisting of: methyl, ethyl, n-propyl, isopropyl, cyclopropyl, cyclopropylmethyl, cyclobutyl and cyclopentyl;
R² is selected from the group consisting of: chloro, methyl, ethyl, propyl, isopropyl and methoxy, and
R³ is selected from the group consisting of: methyl, ethyl, n-propyl and n-butyl.

6. A compound in accordance with claim 1 selected from the group consisting of:
TABLE 1 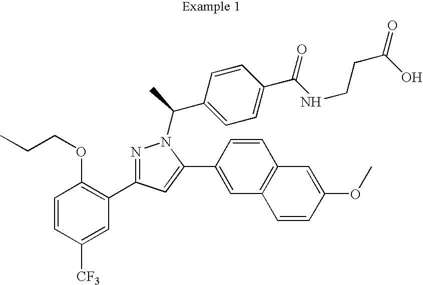 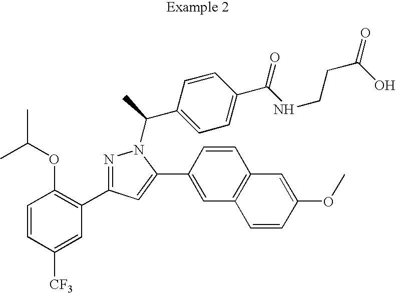 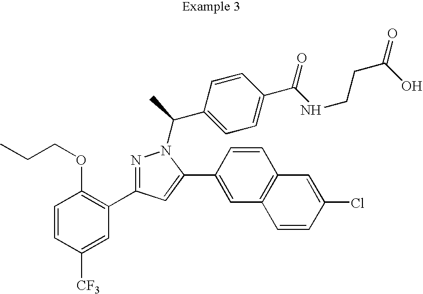 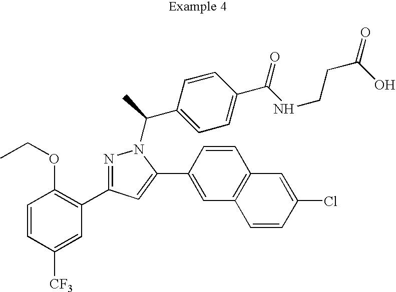 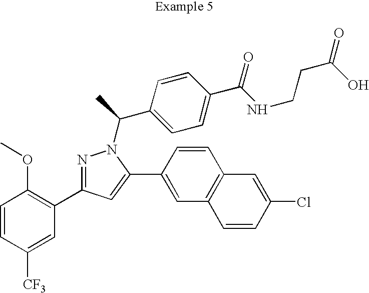 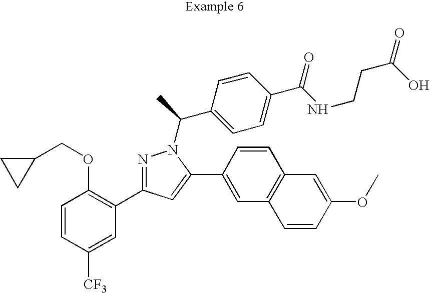 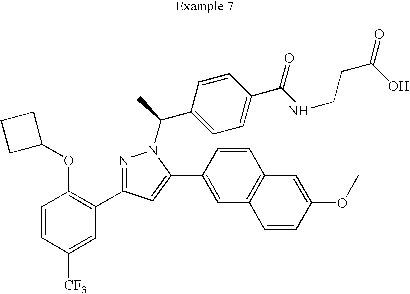 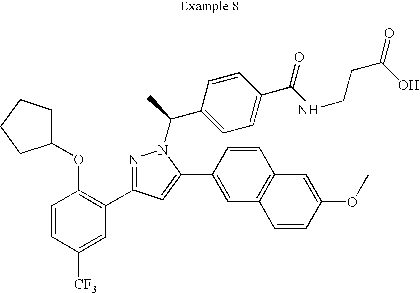 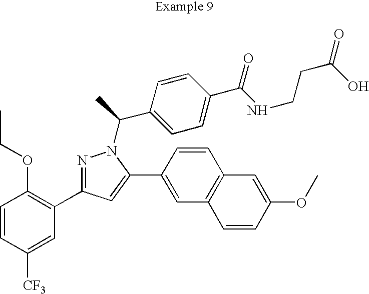 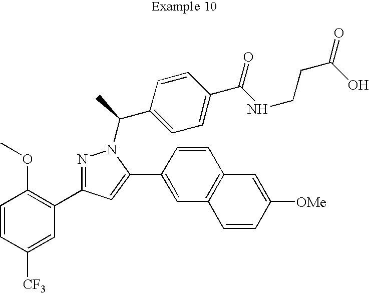 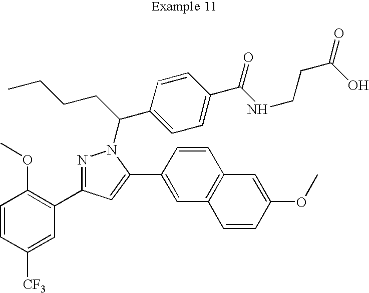 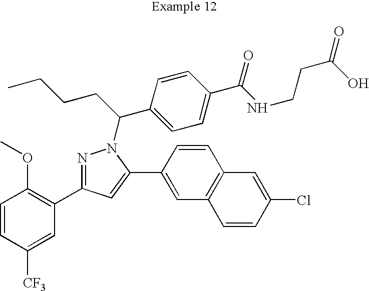 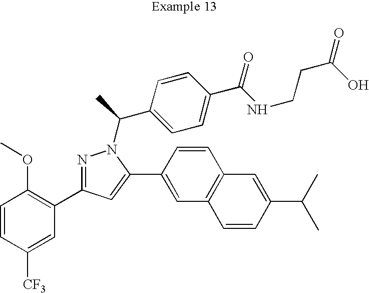
or a pharmaceutically acceptable salt or solvate thereof.

7. A pharmaceutical composition comprising a compound in accordance with claim 1 in combination with a pharmaceutically acceptable carrier.

8. A method of treating type 2 diabetes mellitus in a mammalian patient in need of such treatment comprising administering to said patient a compound in accordance with claim 1 in an amount that is effective to treat said type 2 diabetes mellitus.

9. A method of delaying the onset of type 2 diabetes mellitus in a mammalian patient in need thereof, comprising administering to the patient a compound in accordance with claim 1 in an amount that is effective to delay the onset of said type 2 diabetes mellitus.

10. A method of treating hyperglycemia, diabetes or insulin resistance in a mammalian patient in need of such treatment which comprises administering to said patient an effective amount of a compound in accordance with claim 1.

11. A method of treating non-insulin dependent diabetes mellitus in a mammalian patient in need of such treatment comprising administering to the patient an anti-diabetic effective amount of a compound in accordance with claim 1.

12. A method of treating obesity in a mammalian patient in need of such treatment comprising administering to said patient a compound in accordance with claim 1 in an amount that is effective to treat obesity.

13. A method of treating Syndrome X in a mammalian patient in need of such treatment, comprising administering to said patient a compound in accordance with claim 1 in an amount that is effective to treat Syndrome X.

14. A method of treating a lipid disorder selected from the group consisting of dyslipidemia, hyperlipidemia, hypertriglyceridemia, hypercholesterolemia, low HDL and high LDL in a mammalian patient in need of such treatment, comprising administering to said patient a compound in accordance with claim 1 in an amount that is effective to treat said lipid disorder.

15. A method of treating atherosclerosis in a mammalian patient in need of such treatment, comprising administering to said patient a compound in accordance with claim 1 in an amount effective to treat atherosclerosis.

16. A method of treating a condition selected from the group consisting of: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component, in a mammalian patient in need of such treatment, comprising administering to the patient a compound in accordance with claim 1 in an amount that is effective to treat said condition.
